(19) United States
(12) Patent Application Publication
(10) Pub. No.: US 2014/0025143 A1
(43) Pub. Date: Jan. 23, 2014

(54) DEVICES AND METHODS TO REDUCE MYOCARDIAL REPERFUSION INJURY

(71) Applicant: Prospex Medical III, New Brighton, MN (US)

(72) Inventors: Robert Atkinson, White Bear Lake, MN (US); Jason Galdonik, Minneapolis, MN (US); Peter Keith, Lanesboro, MN (US); Paul McLean, North Oaks, MN (US)

(73) Assignee: Prospex Medical III, New Brighton, MN (US)

(21) Appl. No.: 13/943,605

(22) Filed: Jul. 16, 2013

Related U.S. Application Data
(60) Provisional application No. 61/672,528, filed on Jul. 17, 2012.
(60) Provisional application No. 61/776,399, filed on Mar. 11, 2013.

(51) Int. Cl. A61F 7/12

(52) U.S. Cl. CPC A61F 7/12
USPC 607/105

(57) ABSTRACT

Devices and methods that mitigate reperfusion injury (RI) in a clinically practical manner so as to avoid significantly increasing time to reperfusion. In general, these systems and methods involve an antegrade approach to deliver a fluid to the myocardium at risk of RI before, during and after reperfusion is established by a percutaneous coronary intervention such as aspiration and stenting.

### CROSS-REFERENCE TO RELATED APPLICATIONS

This patent application claims the benefit of U.S. Provisional Patent Application No. 61/672,528, filed Jul. 17, 2012, entitled Devices and Methods to Reduce Myocardial Injury, and U.S. Provisional Patent Application No. 61/776,399, filed Mar. 11, 2013, entitled Devices and Methods to Reduce Myocardial Injury, the entire disclosures of which are incorporated herein by reference.

### FIELD OF THE DISCLOSURE

Embodiments of the present disclosure describe devices and methods that mitigate reperfusion injury (RI) in a clinically practical manner so as to avoid significantly increasing time to reperfusion

### BACKGROUND OF THE DISCLOSURE

S-T segment elevated myocardial infarction (STEMI) occurs when a major coronary artery, typically the left anterior descending artery, is significantly blocked resulting in ischemia of the myocardium of the left ventricle. This results in characteristic changes in an electrocardiogram (ECG) recognized as an elevated S-T segment indicating that a large portion of the heart is being damaged. Due to the size and significance of the affected area, STEMI patients represent the highest risk group of patients presenting with acute myocardial infarction (AMI). The duration of ischemia, or time-to-reperfusion, is a major factor influencing the size of the infarct, which is a major determining factor influencing acute and chronic clinical outcomes (e.g., mortality, left ventricular ejection fraction, cardiac functional capacity, congestive heart failure, etc.).

Current medical guidelines call for rapid reperfusion of the ischemic area through thrombolytic therapy and/or primary percutaneous coronary intervention (PPCI) including balloon angioplasty and stenting. The restoration of blood flow to the affected area is intended to limit the duration of ischemia and reduce the size of the infarct. Clinical trials have demonstrated that the sooner reperfusion is established, the smaller the size of the infarct and the better the clinical outcome, hence the mantra to minimize “door-to-balloon” time. However, restoration of blood flow to the ischemic area can result in additional injury to the affected area. This phenomenon has been termed reperfusion injury (RI).

Reperfusion injury can be defined as dysfunction of the heart induced by restoration of blood flow to a previously ischemic area. There are four main types of dysfunction induced by reperfusion. The first is mechanical dysfunction or reduced contractile function of the left ventricular wall. The second type of dysfunction is termed the no-reflow phenomenon. No-reflow is defined as the impedance of blood flow to the micro vascular structures of the myocardium inhibiting reperfusion of the ischemic area. The third type of dysfunction is arrhythmias induced by the reperfusion. The final component of reperfusion injury is termed lethal reperfusion injury. Lethal reperfusion injury is defined as continued cardiac myocyte (heart muscle) death as a consequence of reperfusion. Lethal reperfusion injury has been shown to contribute to a significant portion (one third or more) of tissue necrosis after ischemia. The mechanisms of lethal reperfusion injury are multifactorial and complex including metabolic, biochemical and cellular responses to both ischemia and reperfusion.

A number of strategies have demonstrated reduction in infarct size post reperfusion in both animal models an in the clinical setting. Although the mechanisms of these strategies are not fully understood, there is a growing body of evidence to suggest they can reduce infarct size.

One of these strategies involves hypothermia, which has been demonstrated to reduce infarct size. Hypothermia involves the reduction of tissue temperature in order to reduce metabolic rate and/or enzymatic activity resulting in protection of the affected tissues. Therapeutic hypothermia as applied to AMI is described in more detail by Hale et al., Mild hypothermia as a cardioprotective approach for AMI: lab to clinical application, 2011.

Whole body cooling, by external and internal means, has been used to induce therapeutic hypothermia. Examples of external cooling devices include ice baths, cold packs and cooling blankets. Examples of internal cooling devices include a balloon catheter placed in the vena cava, where cold fluid is circulated through the balloon to cool the passing blood. In general, whole body cooling systems require a significant amount of time to achieve the desired temperature drop, which is at odds with the effort to minimize door-to-balloon time in STEMI patients.

### SUMMARY OF INVENTION

It is desirable to start therapeutic hypothermia before reperfusion, and it is desirable to minimize time to reperfusion. Thus, a clinically successful hypothermic intervention is preferably performed before reperfusion by PPCI without significantly increasing door-to-balloon time. This represents a significant practical challenge in the clinical setting which has thus far eluded a practical solution. The present invention provides a number of different embodiments to address this challenge.

In general, the present invention provides therapeutic hypothermia systems and methods that may protect the myocardium from reperfusion injury (RI) in a clinically practical manner so as to avoid significantly increasing time to reperfusion. These systems and methods involve an antegrade approach to deliver a fluid to the myocardium at risk of RI before, during and after reperfusion is established by PPCI (e.g., dilating the culprit lesion with a balloon catheter/stent). A variety of devices, fluids, and procedural steps are disclosed to mitigate RI by, for example: pre-conditioning (e.g., reducing the temperature of) the affected myocardium; controlling reperfusion dynamics (e.g., flow rate, oxygenation, flushing, buffering, etc.) to the affected myocardium, and/or post-conditioning the affected myocardium. These systems and methods could be used as a stand-alone therapy, or used to augment other therapeutic hypothermia approaches (e.g., whole body cooling) and other interventional procedures.

In embodiments of the present invention, reducing the temperature of the affected myocardium may reduce the rate of adverse reactions (e.g., toxic oxygen reactions, inflammatory cascades, etc.) associated with RI. Flushing the affected myocardium may reduce the presence of metabolic imbalances and adverse agents (e.g., calcium overload, lactic acid build-up, etc.) associated with RI. Delivering beneficial agents to the downstream vasculature may mitigate vasoconstriction and thrombus formation associated with no re-flow. Controlling reperfusion to the affected myocardium may meter the introduction of reagents (e.g., oxygen) that bring about adverse reactions (e.g., toxic oxygen species) while supporting beneficial reactions (e.g., ATP production).

The embodiments of the present invention are described herein with reference to STEMI patients, where the ischemic myocardium is typically on the anterior side of the left ventricle and is typically caused by a restriction in the left anterior descending artery. However, the principles of the present invention may be applied to other myocardial areas, other coronary arteries, and arterial restrictions in other locations.

Similarly, while specifically useful for treating STEMI, the embodiments of the present invention may be used for other coronary indications such as all emergent or acute coronary syndromes (e.g., acute myocardial ischemia, unstable angina, etc.) and all non-emergent or elective coronary syndromes (e.g., stable angina). In addition, while specifically useful for treating the heart, the embodiments of the present invention may be used with other organs such as the brain (e.g., stroke therapy), lungs (e.g., pulmonary embolism therapy) and kidneys (e.g., renal failure).

In one embodiment, a cold fluid may be delivered via an infusion device extending through a guide catheter and across the culprit restriction in an artery. The device may comprise an infusion guide wire, an infusion catheter, an embolic protection (capturing) device, a balloon catheter, or a stent delivery catheter, for example, each with a lumen to transport the cold fluid. The infusion device may be configured to be compatible with conventional PPCI hardware (e.g., guide catheters, guide wires, thrombus removal catheters, balloon catheters, stent delivery catheters, etc.), and may be configured to maximize uninterrupted cooling during the PPCI procedure.

With the infusion device positioned distally of the culprit restriction, the cold fluid may be administered before the restriction is opened. Although the act of crossing the restriction with the infusion device may partially open the restriction, the cold fluid may be administered before the restriction is fully dilated. Optionally, an occlusion balloon may be provided on the distal end of the infusion device to occlude or reduce blood flow in the artery while the cold fluid is being delivered. Delivery of the cold fluid may be maintained during reperfusion and sustained for a period of time thereafter.

The cold fluid may comprise, for example, a crystalloid solution (e.g. saline), a lactate solution (e.g., Ringer's), a radiopaque contrast solution used for angiographic visualization, autologous or non-autologous oxygenated (e.g., arterial) blood, autologous or non-autologous low-oxygenated (e.g., venous) blood, and/or a combination thereof. It may be desirable to control the rate of oxygen delivery to the affected myocardium to mitigate reperfusion injury. Accordingly, the cold fluid may have a lower oxygen content than arterial blood and/or may be delivered at a slower flow rate to reduce the rate of oxygen delivery relative to normal arterial blood flow. Reducing the rate of oxygen delivery to the affected myocardium is intended to provide a basis for modest ATP production while minimizing toxic oxygen reactions. By providing a small amount of oxygen to the affected myocardium, vital ATP may be produced at a ratio of 32:1, while toxic reactants may be produced at a much lower ratio. In one example, the cold fluid is arterial blood, but delivered at a flow rate that is well below normal. In another example, the cold fluid is crystalloid or the like, which has a much lower oxygen content than arterial blood, and can be delivered at any physiological flow rate. In another example, the cold fluid is a mix of the two, the mix ratio of which can be fixed or varied over the treatment time. In each of these examples, the ischemic myocardium is receiving some oxygen but less than that provided by restored normal blood flow across the restriction after dilation by PPCI.

Delivery of the cold fluid may be continued until a target temperature (e.g., 32 C-35 C) is achieved in the affected myocardium. The target temperature may be achieved before reperfusion is established across the culprit restriction, maintained during reperfusion, and sustained for a period of time thereafter. The temperature of the affected myocardium may be indirectly measured using a thermal sensor (e.g., thermocouple) on a distal end of the infusion device, guide catheter or other device. Alternatively the temperature could be measured using a thermal sensor placed in the liquid before it is delivered into the body, such as in the hub of the infusion device, in an accessory such as a stop cock or hemostasis valve assembly, or in the tubing connecting the infusion device to a pump/cooler. The temperature of the affected myocardium may be estimated, for example, by applying the temperature measurement from the temperature sensor to an algorithm based on an empirically established heat transfer model or a thermodynamic model of the heart and coronary vasculature that assumes a given blood flow rate in the artery (e.g., TIMI flow score).

Various other embodiments of the present disclosure are described in the following detailed description and referenced drawings.

### BRIEF DESCRIPTION OF THE DRAWINGS

The drawings illustrate example embodiments of the present invention. The drawings are not necessarily to scale. Similar elements in different drawings may be numbered the same. In the drawings:

FIG. 1 is an anterior view of a human heart illustrating the coronary arteries with a restriction in the left anterior descending artery affecting the downstream myocardium;

FIGS. 2A and 2B are a schematic flow chart and plot showing general methods of the present invention;

FIG. 3 is a schematic flow diagram of a typical primary percutaneous coronary intervention (PPCI) procedure showing the timing of pre-conditioning according to the present invention;

FIGS. 4 and 5 are schematic diagrams of systems for delivering a cold fluid to the left main coronary artery via a guide catheter;

FIGS. 6 and 7 are schematic diagrams of systems for delivering a cold fluid to the left anterior descending artery distal of a restriction via a crossing device;

FIGS. 8 and 9 are schematic diagrams of systems for delivering cold fluids to both the left main coronary artery and the left anterior descending artery distal of a restriction.

FIG. 10 is a schematic flow diagram of a modified PPCI procedure showing steps for using the system shown in FIG. 9;

FIG. 11 is a schematic drawing of a guide catheter for use in any of the systems shown in FIGS. 4-9;

FIGS. 12 and 13 are schematic diagrams of a crossing device in the configuration of an infusion guide wire;

FIGS. 14 and 15 are schematic diagrams of a crossing device in the configuration of an infusion catheter;

FIGS. 16 and 17 are schematic diagrams of a crossing device in the configuration of a balloon catheter;

FIGS. 18 and 19 are schematic diagrams of a crossing device in the configuration of a stent delivery catheter;

FIGS. 20-50 are schematic illustrations of various intravascular devices that may be used in an antegrade hypothermia system; and

FIGS. 51-53 illustrate empirically derived thermal loss data in different models.

### DETAILED DESCRIPTION OF THE DISCLOSURE

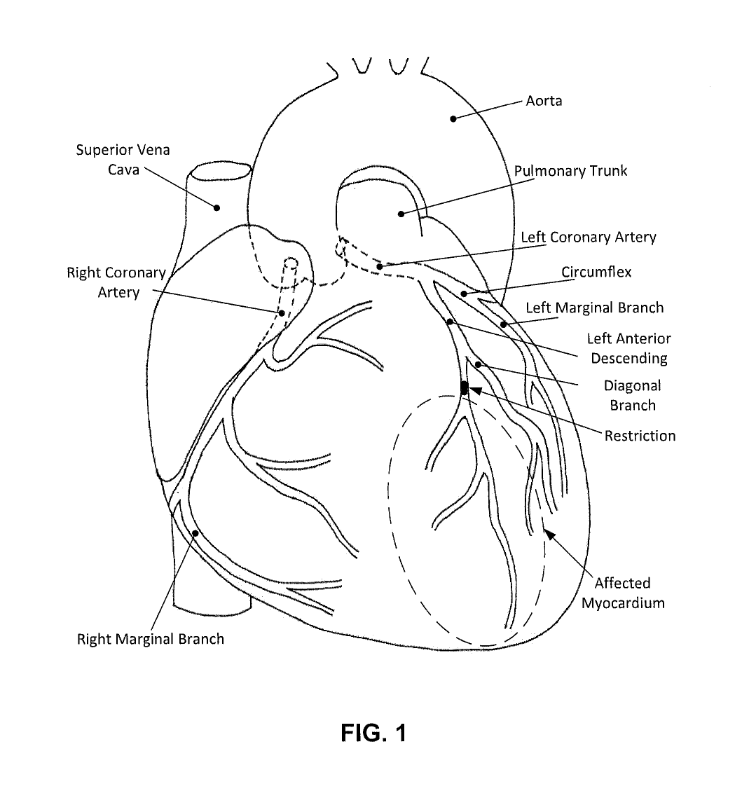

With reference to FIG. 1, an anterior view of a human heart is shown. The heart includes two main coronary arteries: the right coronary and the left coronary artery. The right coronary artery generally provides oxygenated blood to the myocardium of the right atrium and ventricle, and the left coronary artery generally provides oxygenated blood to the myocardium of the left atrium and ventricle. Major branches of the right coronary artery include the right marginal branch, and major branches of the left coronary artery include the left anterior descending, the circumflex, the left marginal (branch of circumflex), and the left diagonal (branch of left anterior descending). Patients with ST elevation myocardial infarction (STEMI) often have a restriction in the left anterior descending artery affecting the downstream myocardium as shown. Although the present invention is described with reference to this presentation of STEMI, the principles of the present invention may be applied to other clinical presentations, other myocardial areas, other coronary arteries, and arterial restrictions in other locations.

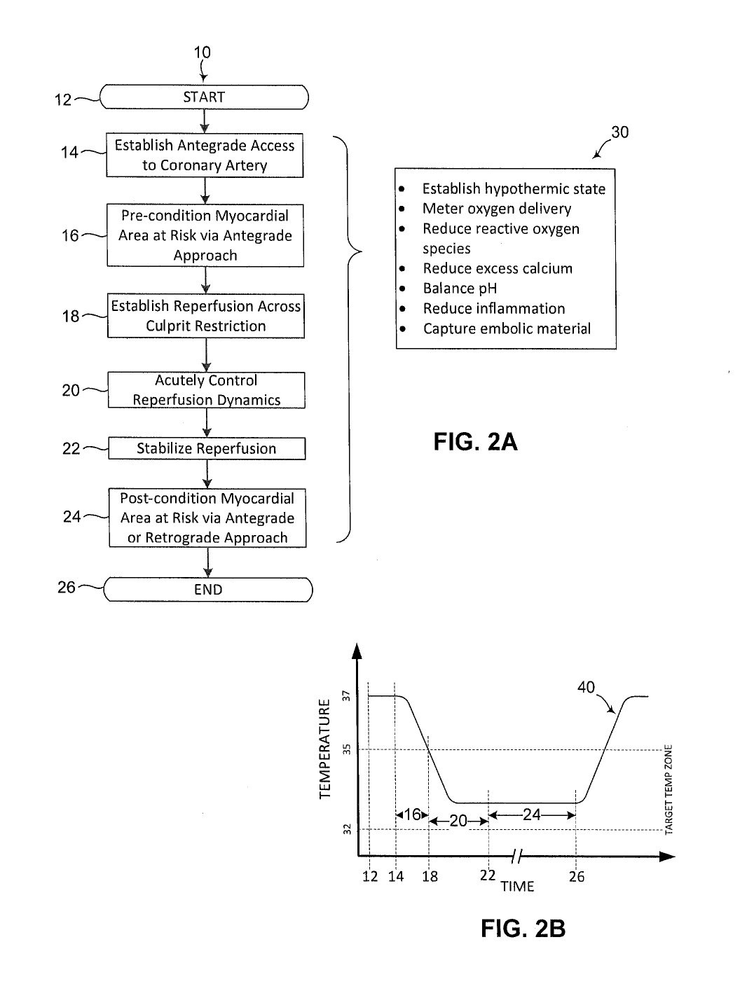

With reference to FIG. 2A, general steps of a method 10 for mitigating reperfusion injury according to an embodiment of the present invention are shown schematically with reference to general objectives 30. The method 10 may be broken down into three generalized steps (16, 20, 24) relative to the time at which antegrade coronary artery access is established 14, relative to the time reperfusion across the culprit restriction is established 18, and relative to the time stabilized reperfusion is established 22. The myocardial temperature 40 relative to each step is graphically represented by the plot in FIG. 2B, which is representative but not necessarily to scale.

The step 14 of accessing to the coronary artery may be accomplished using a guide or diagnostic catheter as is common in PCI procedures. Access to the coronary artery typically represents the first instance where focal cooling may be administered using conventional steps in PCI. Immediately after or coincident with establishing access 14, the affected myocardium or myocardium at risk may be focally pre-conditioned 16 via the established antegrade access.

The step of pre-conditioning 16 may involve establishing a mild hypothermic state in the affected myocardium at a temperature below normal body temperature (37 C) but above a temperature associated with adverse cardiac effects such as arrhythmia. Some clinical literature sources report no beneficial effect at 36 C, but significant beneficial effect at or below 35 C. Other clinical literature sources report adverse cardiac events below 32 C. Thus, the target temperature zone may be 32 C to 35 C. However, to the extent that the hypothermic state is localized to only a portion of the myocardium (as opposed to the whole heart and/or the whole body), it may be safe to target a myocardial temperature below 32 C. Thus, the target myocardial temperature zone of 32 C-35 C as shown in FIG. 2B is provided by way of example, not necessarily limitation.

The pre-conditioning 16 may also involve flushing adverse agents (reactive oxygen species, excess calcium, lactic acid, inflammatory agents) from the affected myocardium and/or delivering beneficial agents (vasodilators, thrombolytics, etc.) to the downstream vasculature. This may be accomplished, for example, by delivering a cold fluid via the guide or diagnostic catheter seated in the coronary artery. Alternatively or in addition, this may be accomplished by delivering a cold fluid via a crossing device extending across the culprit restriction. Reducing the temperature of the affected myocardium is intended to reduce the rate of adverse reactions (e.g., toxic oxygen reactions, inflammatory cascades, etc.) associated with reperfusion injury. Flushing the affected myocardium is intended to reduce the presence of metabolic imbalances and adverse agents (e.g., calcium overload, lactic acid build-up, etc.) associated with reperfusion injury. Delivering beneficial agents to the downstream vasculature is intended to mitigate vasoconstriction and embolic formation associated with no re-flow after reperfusion. The step of focal pre-conditioning 16 may begin as soon as possible after coronary artery access 14 is established in order to reach the myocardial target temperature zone before reperfusion 18 is established as shown in FIG. 2B. Also as shown in FIG. 2B, reperfusion across the culprit restriction 18 may be initiated once the myocardial temperature 40 is within the target zone. The myocardial temperature 40 may be maintained within the target zone from a time prior to or coincident with reperfusion 18 through the end 26 of post-conditioning 24, at a constant or minimally fluctuating manner as seen in FIG. 2B.

The step of controlling reperfusion dynamics 20 may involve continuing the pre-conditioning measures 16 as well as metering oxygen delivery to the affected myocardium by controlling the flow rate and/or oxygen concentration in the cold fluid being delivered past the restriction. Reperfusion injury may begin immediately upon establishing reperfusion 18, and may be most significant in the first 5 minutes thereafter. Therefore, it may be desirable to control reperfusion dynamics 20 coincident with but no greater than 5 minutes after the initiation of reflow across the restriction 18. Reperfusion 18 may occur when the restriction is aspirated, dilated by a balloon catheter, and/or dilated by a stent delivery catheter. Thus, whichever device (guide wire, thrombus removal catheter, balloon catheter, or stent delivery catheter) is used to establish the first instance of reperfusion 18, it may be desirable to configure that device to control reperfusion dynamics 20. In general, the step of controlling reperfusion dynamics 20 to the affected myocardium is intended to meter the introduction of reagents (e.g., oxygen) that bring about adverse reactions (e.g., toxic oxygen reaction) while supporting beneficial reactions (e.g., ATP production).

Controlling reperfusion dynamics 20 may also involve embolic protection using a known emboli capturing device deployed downstream (distal) of the restriction. Embolic material captured during reperfusion and after reperfusion may be aspirated using known thrombus removal catheters.

The step of post-conditioning 24 may involve continuing the pre-conditioning measures 14 and/or the reperfusion measures 18. For example, it may be desirable to continue mild hypothermia, continue flushing, continue medication, and/or continue metering oxygen. This may be done after PCI is complete and stable reflow is established 22. For example, to facilitate post-conditioning 24, the guide catheter may remain in place in the coronary artery through which cold fluid may continue to be administered. In addition or as an alternative, the crossing device may remain in place across the dilated restriction through which oxygen may continue to be metered. This may be initiated in the cath lab and continued in the recovery room, for example, and may last 30 to 120 minutes. Because the typical recovery room is not equipped with angiography capability, it may be desirable to incorporate an anchoring balloon on the distal end of the guide catheter and/or the crossing device to stabilize the same in the coronary artery. To avoid thrombus formation in the guide catheter and/or crossing device, it may be desirable to continuously flush the same with the either the cold oxygen controlled fluid or a neutral fluid until the device is removed. If desired, the step of post-conditioning 24 may be performed with a retrograde approach via the coronary sinus. After the post-conditioning 24 is complete 26, the myocardium may be allowed to return to normal temperature as shown in FIG. 2B.

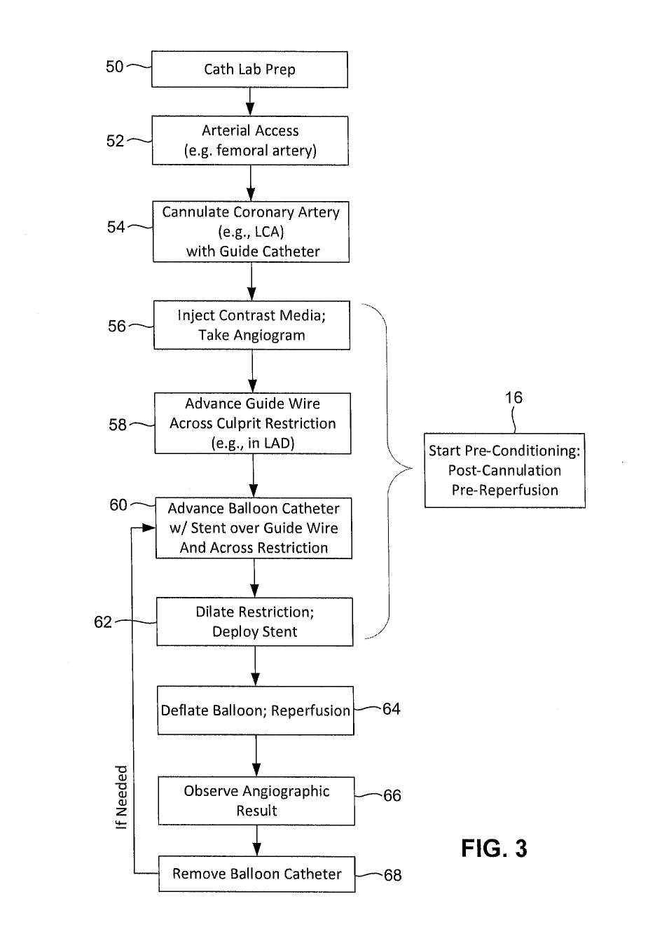

With reference to FIG. 3, the basic steps involved in a typical primary percutaneous coronary intervention (PPCI) procedure are shown in a flow chart. Once a patient has been diagnosed as having a STEMI and the cath lab personnel have been notified, the cath lab is prepared 50 to receive the patient. Once the patient is in the cath lab and prepped, a needle, wire and access sheath are used to establish arterial access, usually in the femoral artery or radial artery. A guide (or diagnostic) catheter is then routed 54 to the right or left coronary artery and radiopaque contrast media is injected 56 into the coronary artery via the catheter while taking x-ray images to produce an angiogram. Once the culprit restriction is identified by angiography, a guide wire is advanced 58 through the guide catheter until its distal end extends across the restriction. A balloon and/or stent delivery catheter is then advanced 60 over the guide wire to position the balloon and/or stent across the restriction. The restriction is then dilated 62 and/or a stent is deployed. Once the balloon is deflated 64, reperfusion is established across the restriction. An angiogram is then taken to observe 66 flow across and downstream of the dilated restriction. In some instances, the vasculature distal of the dilated restriction will appear to have little or no flow, sometimes referred to as “no re-flow” phenomenon. In such cases, medication such as a vasodilator may be administered through the guide catheter to open the distal vascular bed. The balloon or stent delivery catheter is then removed 68, and if needed, additional stents may be deployed 60.

The pre-conditioning steps 16 may start any time after cannulation 54 of the coronary artery and before deflating 64 the balloon to establish reperfusion, i.e., post-cannulation to pre-reperfusion. Since significant reperfusion injury occurs in the first 5 minutes after reperfusion is initiated 64, it may be desirable to allow sufficient time for pre-conditioning measures to prepare the affected myocardium for reperfusion. As such, it may be desirable to start pre-conditioning immediately after cannulation 54 of the coronary artery thus maximizing procedural time for pre-conditioning measures such as hypothermia to have effect.

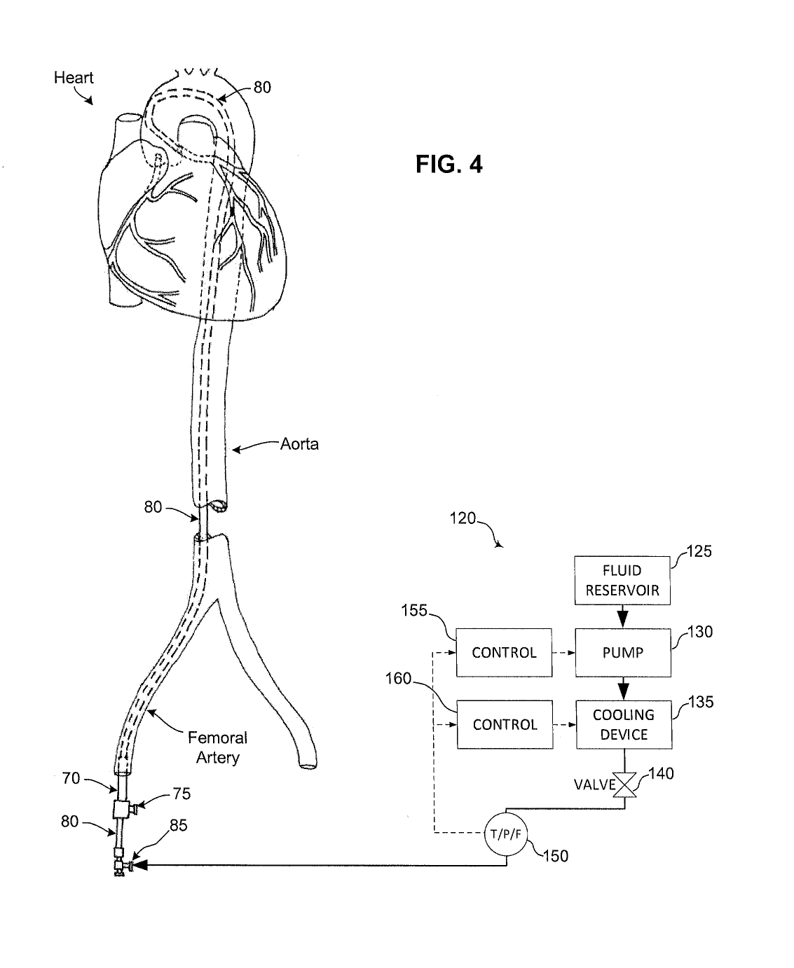

With reference to FIG. 4, a system 120 for administering local hypothermia is shown schematically. The system 120 delivers a cold fluid to the coronary artery via a guide catheter 80. Because some physicians prefer to make periodic intravascular pressure measurements via the guide catheter 80, a valve 140 positioned proximal of the guide catheter 80 may be used to periodically stop infusion of the cold fluid, allowing intravascular pressure to be measured via a pressure sensor 150 positioned distal of the valve 140.

An access sheath 70 is percutaneously positioned in a peripheral artery such as the radial or femoral artery as shown. The guide catheter 80 extends through the access sheath 70, up the descending aorta, over the aortic arch, down the ascending aorta, with its distal end seated in the ostium of the right or left main coronary artery as shown. Both the vascular access sheath 70 and the guide catheter 80 include a hub or manifold 75, 85 (respectively) allowing coaxial insertion of devices into the internal lumen thereof, and allowing infusion of fluids through a side port in fluid communication with the internal lumen.

The system 120 may include a fluid reservoir 125 for holding non-autologous fluid such as saline, blood, plasma, Ringer's lactate, or any other fluid suitable for intravascular injection. Oxygenated blood is beneficial because it can support the metabolic demands of the occluded artery proximal of the restriction as well as non-occluded arteries. The fluid may contain additives such as blood thinning agents, anti-inflammatory agents, vaso-dilators, anti-platelet agents, buffering agents, potassium, glucose, oxygen, or other beneficial additives and pharmacological agents. The fluid contained in reservoir 125 is pressurized by pump 130, which may be a volume-controlled or pressure-controlled pump, for example, and may pump fluid in a constant or pulsatile manner. Pressurized fluid leaves the pump 130 and enters a cooling device 135, which may comprise a heat exchanger such as a refrigerant device, Peltier-effect device, ice bath, etc. Optionally, the cooling device 135 may be incorporated into the reservoir 125 or the pump 130. An interface such as disposable tubes and/or a disposable cartridge may be utilized to contain the fluid, leaving the remaining portions (e.g., 125, 130, 135) of the system 120 reusable. Pressurized and cooled fluid then enters the guide catheter 80 through its side port 85, optionally via a control valve 140. Pressurized and cooled fluid passes through a lumen in the guide catheter 80 and exits the distal end thereof, thus localizing cooled fluid to the coronary artery.

Proximal sensor or sensors 150 may be used to measure temperature, pressure and/or flow. The sensors 150 may be functionally linked via wires (dashed lines) to a pump control 155 and a cooling device control 160 to provide feedback control of the pump 130 and cooling device 135, respectively.

As will be described in more detail later, the guide catheter 80 may be equipped with one or more distal sensors. For example, the distal sensor may comprise a temperature sensor (e.g., thermocouple) disposed on the exterior surface of the guide catheter 80 and functionally linked to the cooling device controller 160 via wires extending through the shaft of the guide catheter. In this manner, the temperature of the fluid entering the guide catheter 80 may be adjusted to achieve the desired temperature as measured at the distal end of the catheter in the coronary artery. Such control may be open loop allowing for manual adjustment or closed loop allowing for automatic adjustment of temperature. Optionally, a distal biasing member (e.g., extensible wire loop, not shown) may be incorporated onto the exterior of the distal end of the guide catheter 80 to force the temperature sensor against the arterial wall, which may be more representative of myocardial temperature in the affected area.

Alternatively or in addition, the one or more distal sensors may include a pressure sensor (e.g., pressure transducer) functionally linked to the pump controller 155 via wires. While the pressure sensor 150 connected to the proximal end of the guide catheter 80 may approximate the pressure in the coronary artery after accounting for pressure loss due to head pressure in the catheter 80 and the driving pressure from the pump 130, a more direct pressure measurement may be made with a distal pressure sensor. In this manner, the pressure of the fluid entering the guide catheter 80 may be adjusted to achieve the desired pressure or correlated flow rate at the distal end of the guide catheter in the coronary artery. Such control may be open loop allowing for manual adjustment or closed loop allowing for automatic adjustment of pressure and correlated flow rate. The pressure sensor feedback may also be used as a safety to avoid over-pressurizing the coronary artery.

Alternatively or in addition, the one or more distal sensors may include a flow sensor (e.g., anemometer) to more directly measure and control flow rate. Such control may be open loop allowing for manual adjustment or closed loop allowing for automatic adjustment of pressure and correlated flow rate. The flow sensor feedback may also be used as a safety to avoid over-filling the coronary artery.

The temperature of the cooled and pressurized fluid may be selected to reach a target tissue temperature of 32 C-35 C, for example, within a specified period of time, such as 5-15 min. To achieve a myocardial temperature of 32 C-35 C within the desired timeframe, the cooled fluid may be below 32 C-35 C to establish a sufficient temperature gradient for heat transfer. Given the delicate balance of temperature management at the treatment site, a localized temperature sensor as described previously may be advantageous.

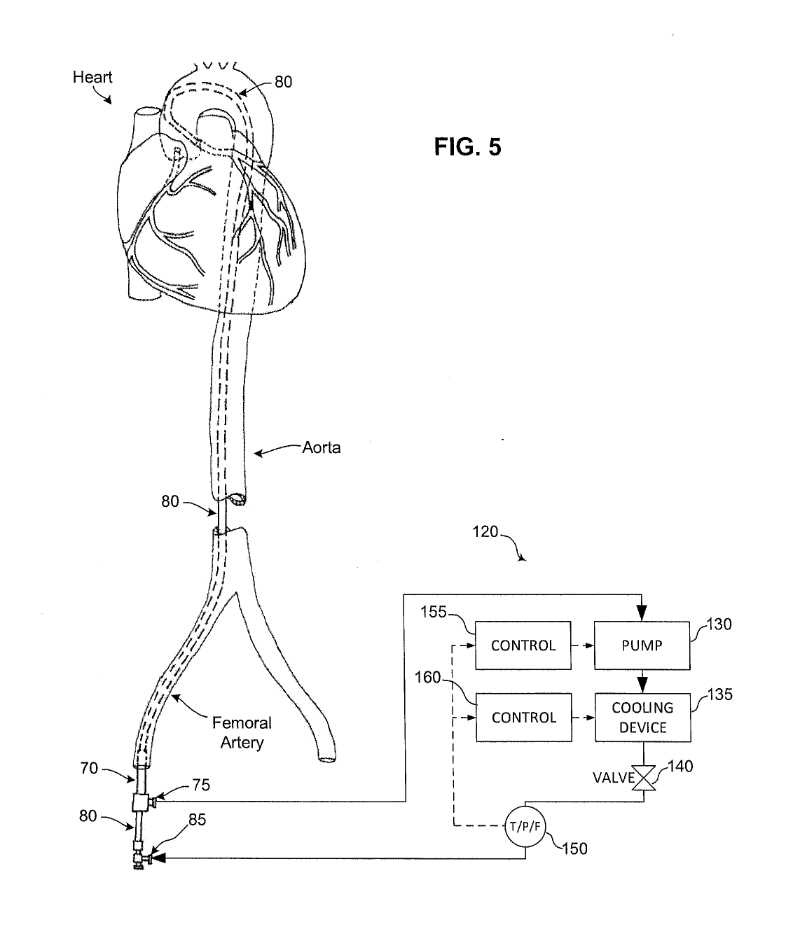

With reference to FIG. 5, an alternative configuration of system 120 is shown schematically. In this embodiment, rather than using a non-autologous fluid from a reservoir, autologous blood is harvested via a blood vessel. In the illustrated example, autologous arterial blood is harvested from the femoral artery via the side port 75 of the access sheath 70, which flows to the pump 130 by suitable tubing. Autologous blood harvested by via access sheath 70 is pressurized by the pump 130 and flows to the cooling device 135 and into the guide catheter 80. Pressurized and cooled autologous blood passes through a lumen in the guide catheter 80 and exits the distal end thereof and into the coronary artery, thus providing localized hypothermia.

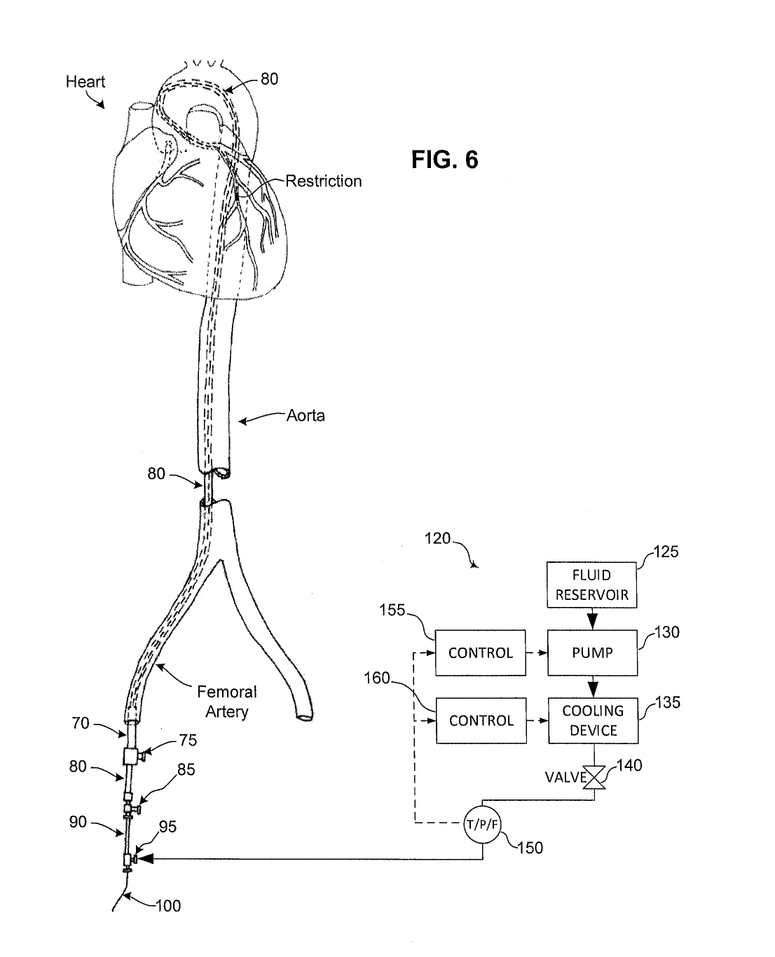

With reference to FIG. 6, an alternative arrangement of system 120 is shown schematically. In this arrangement, rather than delivering the cooled fluid via guide catheter 80, cooled fluid is delivered distal of the restriction by a crossing device 90, which may be advanced over a conventional guide wire 100. Crossing device 90 may be configured as an infusion guide wire, an infusion catheter, an embolic protection (capturing) device, a balloon catheter, or a stent delivery catheter, for example, each with a lumen to transport the cold fluid. Examples of suitable crossing devices are shown and described in more detail later. Fluid from the reservoir 125 is pressurized by the pump 130 and flows to the cooling device 135 and into the crossing device 90 via side port 95. Pressurized and cooled fluid passes through a lumen in the crossing device 90 and exits the distal end thereof. With the distal end of the crossing device extending across the restriction, cooled fluid may be delivered distal of the restriction and directly to the affected myocardium, thus providing localized hypothermia. Alternatively, in this and other embodiments of system 120 where a crossing device 90 is utilized, cooled fluid for the pre-conditioning step 16 may delivered via the crossing device 90, but may be later delivered via the guide catheter 80 for the post conditioning step 24, where there may be a desire to infuse fluid at a higher flow rate. The larger lumen of the guide catheter 80 may be more suitable for delivery of a higher flow rate.

The pump 130, cooling device 135 and control circuits 155, 160 may be housed in a re-usable console (not shown), and the fluid reservoir may be contained in a single-use disposable cartridge the fits into the console. The fluid lines connecting the infusion catheter 90 to the console may be insulated to minimize thermal loss, and the fluid cartridge may be stored in a refrigerator just above the freezing point of the fluid. Just before infusing cold fluid, the fluid lines may be disconnected from the infusion catheter 90 and purged by turning on the pump. This reduces the amount of fluid in the lines that may have warmed during setup. Alternatively, the cold fluid may be continuously recirculated through the pump and cooler using a fluid line loop, and the infusion catheter may tap into the loop.

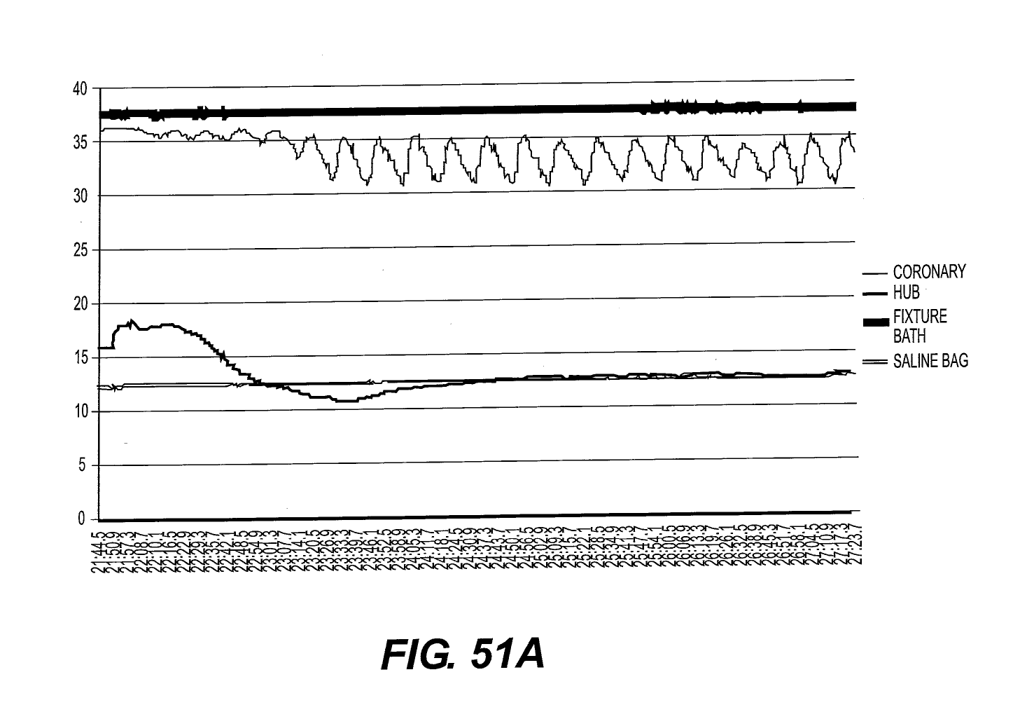

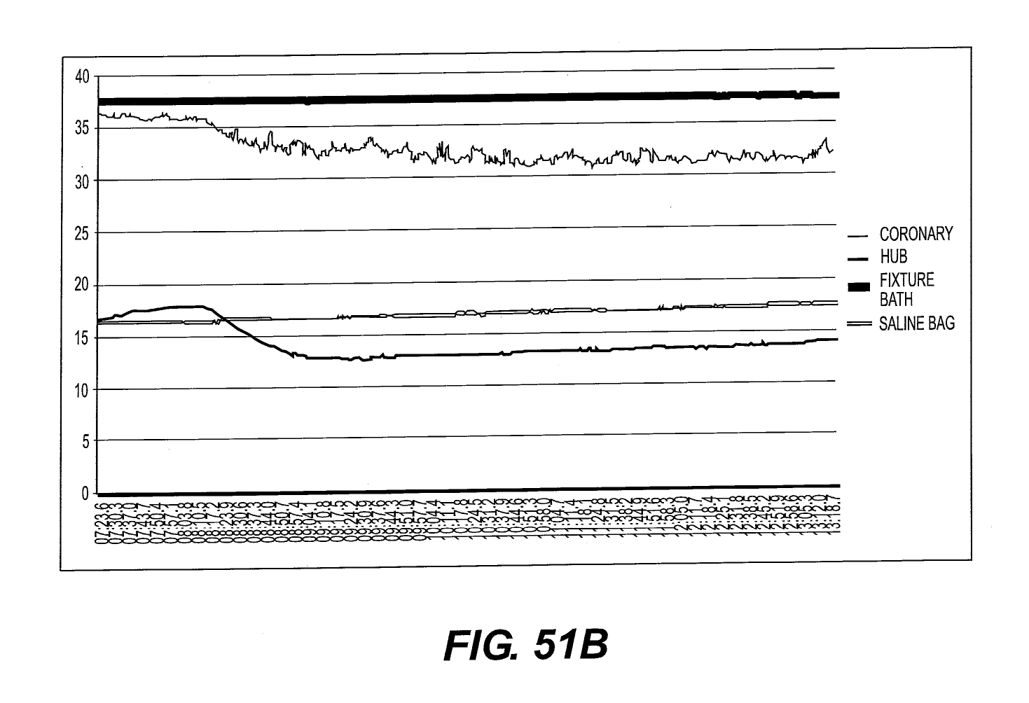

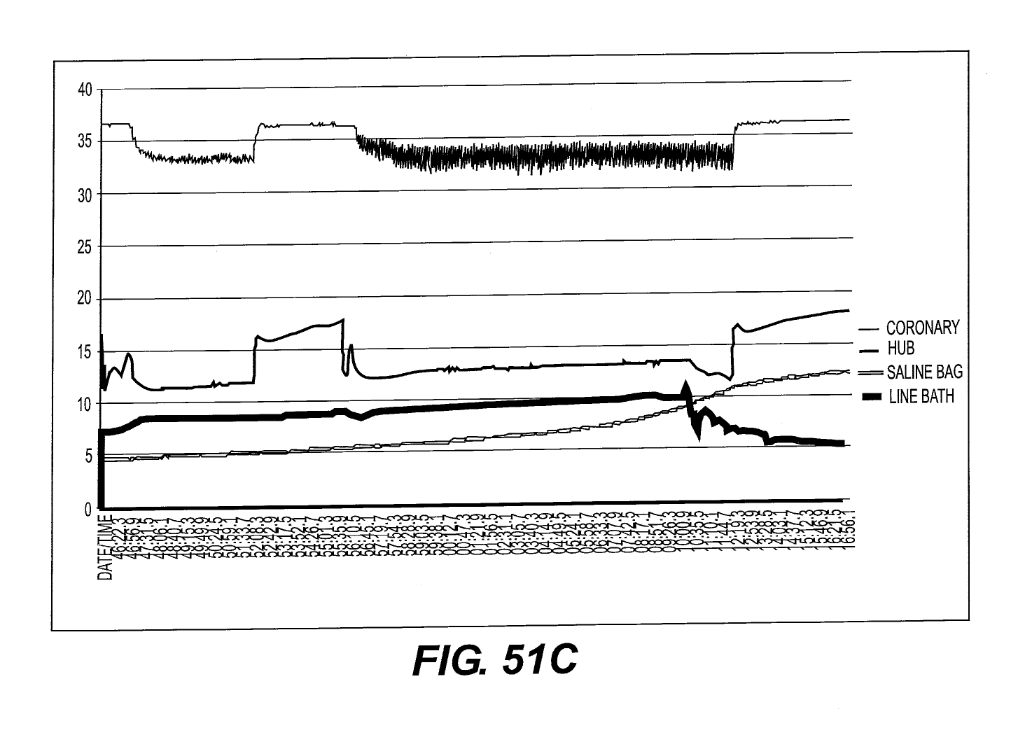

The temperature of the affected myocardium may be estimated, for example, by applying the temperature measurement from the temperature sensor 150 to an algorithm based on an empirically established heat transfer model or a thermodynamic model of the fluid lines, infusion catheter, heart and coronary vasculature for different infusion rates, infusion catheter sizes, blood flow rates in the culprit artery (e.g., TIMI flow score). Examples of such empirically established models are described with reference to FIG. 51 et seq.

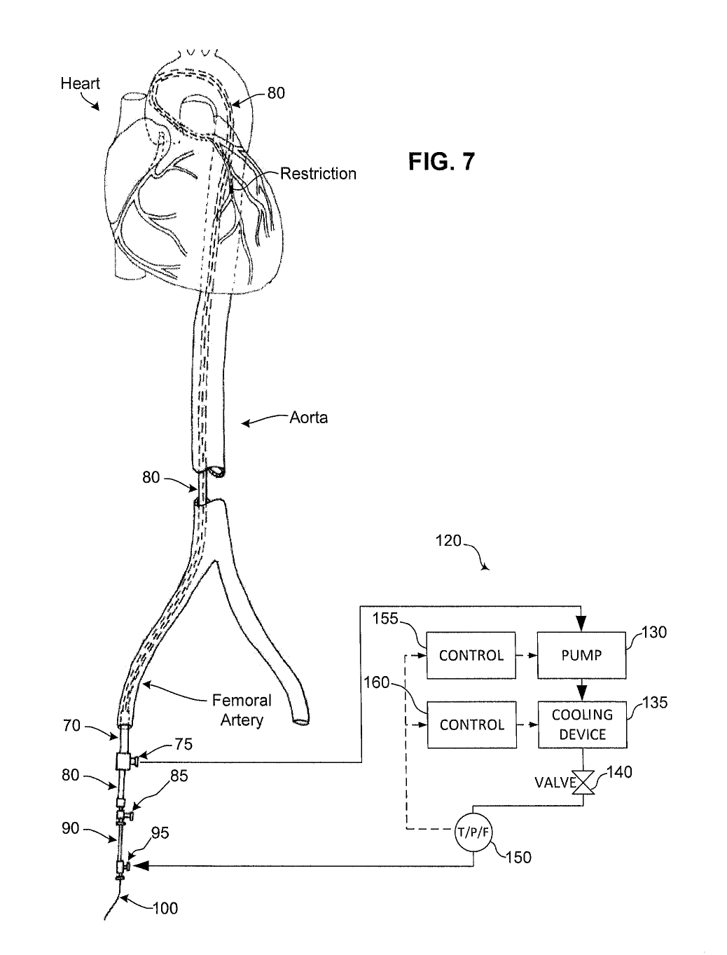

With reference to FIG. 7, a similar alternative arrangement of system 120 is shown schematically. In this arrangement, autologous arterial blood is harvested from the femoral artery via the side port 75 of the access sheath 70, which flows to the pump 130 by suitable tubing. Autologous blood harvested by via access sheath 70 is pressurized by the pump 130 and flows to the cooling device 135 and into the crossing device 90 via side port 95. Pressurized and cooled autologous blood passes through a lumen in the crossing device 90 and exits the distal end thereof distal of the restriction.

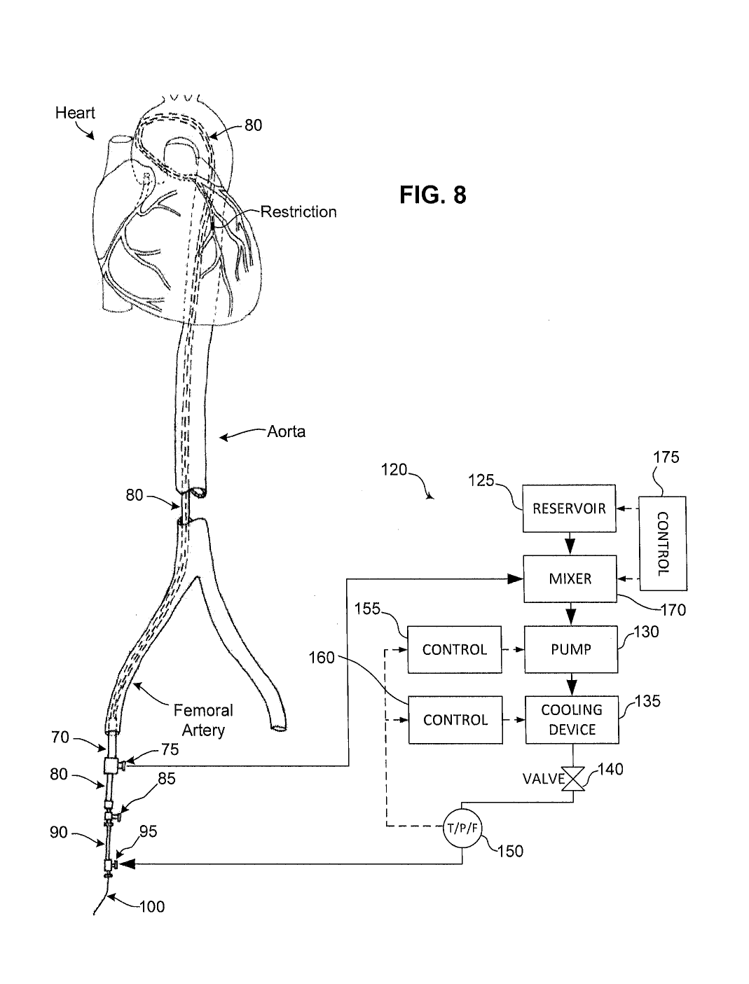

With reference to FIG. 8, an alternative configuration of system 120 is shown schematically. In this configuration, the system 120 may be the same as described earlier, but may include a mixer 170 to facilitate delivery of a mixture of a non-autologous fluid from reservoir 125 and autologous arterial blood from access sheath 70. This configuration may be helpful to deliver a cold fluid distal of the restriction via crossing device 90 as described earlier, while adjusting the oxygen content of the mix by adjusting the mix ratio of non-autologous lower-oxygen content fluid (e.g., crystalloid) from reservoir 125 with autologous higher-oxygen content fluid (e.g., arterial blood) from access sheath 70. A controller 175 may be used to adjust the mix ratio, which may be fixed or variable over the treatment time. For example, a mixed fluid that has a relatively lower oxygen content than arterial blood may be delivered during the first few minutes of reperfusion to minimize the reaction of toxic reactive oxygen species. Subsequently, a mixed fluid that has a relatively normal oxygen content may be delivered after the reactive oxygen species have been substantially flushed from the affected myocardium.

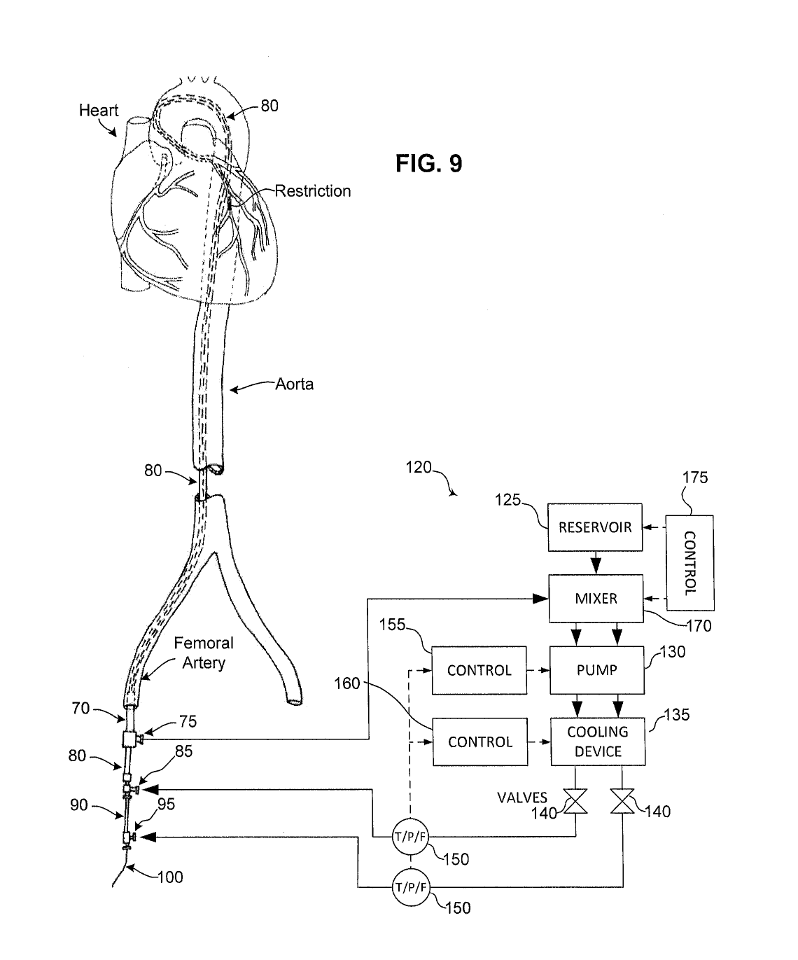

With reference to FIG. 9, another alternative configuration of system 120 is shown schematically. In this configuration, the system 120 may be the same as described with reference to FIG. 8, but may be adapted to deliver two separate fluids or a mix thereof independently via guide catheter 80 and crossing device 90. The first fluid may be sourced from the access sheath 70 and the second fluid may be sourced from the reservoir 125. The first and second fluids may be different types of fluid as described previously, and/or may have different parameters (e.g., temperature, pressure, and/or flow) by independent routing through pump 130, cooling device 135 and valves 140. For example, the first fluid may comprise a cold oxygenated fluid (e.g., arterial blood) delivered at a relatively high flow rate through guide catheter 80, and the second fluid may comprise a cold low- or de-oxygenated fluid (e.g., crystalloid) delivered at a relatively low flow rate through crossing device 90. The first fluid may be gradually mixed into the second fluid to gradually increase its oxygen content as described previously. This arrangement of system 120 facilitates tailored fluid delivery at each stage of pre-reperfusion, intra-reperfusion and post-reperfusion conditioning, both proximal and distal of the restriction.

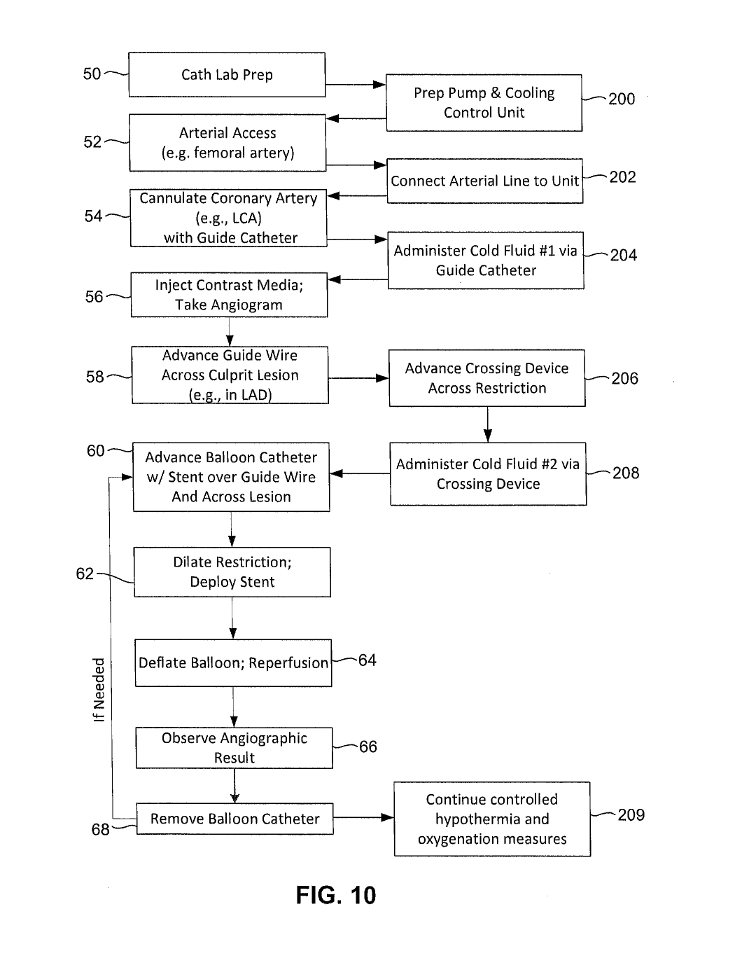

With reference to FIG. 10, a flow diagram shows how a PPCI procedure may be modified to use the system 120 described with reference to FIGS. 9 and 10. When cath lab is being prepared 50 to receive the patient, and the system 120 is prepared 200 for use, including flushing all lines with a liquid to eliminate gas (e.g., air). Arterial access is then established 52, usually in the femoral artery or radial artery, and an arterial line is connected 202 from the access sheath 70 to the system 120. The contrast media and saline mix may also be connected to the system 120 for cooling the same before it is used for angiography. A guide (or diagnostic) catheter 80 is then routed 54 to the right or left coronary artery. Radiopaque contrast media is then injected 56 into the coronary artery via the catheter 80 while taking x-ray images to produce an angiogram. The radiopaque contrast media may be delivered in a cold state by routing first through system 120 as a pre-conditioning measure. Once the culprit restriction is identified by angiography, cold arterial blood (instead of cold contrast media) may be delivered via the guide catheter 80 as a pre-conditioning measure. A guide wire 100 may then be advanced 58 through the guide catheter 80 until its distal end extends across the restriction. A crossing device 90 is then advanced 206 over the guide wire 100 until its distal end extends across the restriction and radiopaque contrast may be used to confirm. If the crossing device 90 is configured as a guide wire, then the crossing device 90 may be used in place of the conventional guide wire 100. Cold fluid (e.g., low oxygen content crystalloid) may then be administered 208 distal of the restriction via crossing device 90 as a pre-conditioning measure. A balloon and/or stent delivery catheter may then be advanced 60 over the guide wire to position the balloon and/or stent across the restriction. The restriction may then be dilated 62 and/or a stent is deployed. Once the balloon is deflated 64, reperfusion is established across the restriction. To control reperfusion dynamics, the administration of cold fluid through the guide catheter 80 and/or the crossing device 90 may include a low- or non-oxygenated fluid. An angiogram may then taken to observe 66 flow across and downstream of the dilated restriction. If a no-reflow condition is observed, medication such as a vasodilator may be administered through the guide catheter 80 and/or crossing device 90 to open the distal vascular bed. The balloon or stent delivery catheter is then removed 68, and if needed, additional stents may be deployed 60. Throughout and after the modified PPCI procedure, controlled hypothermia and oxygenation measures may be continued 209 consistent with the teachings herein.

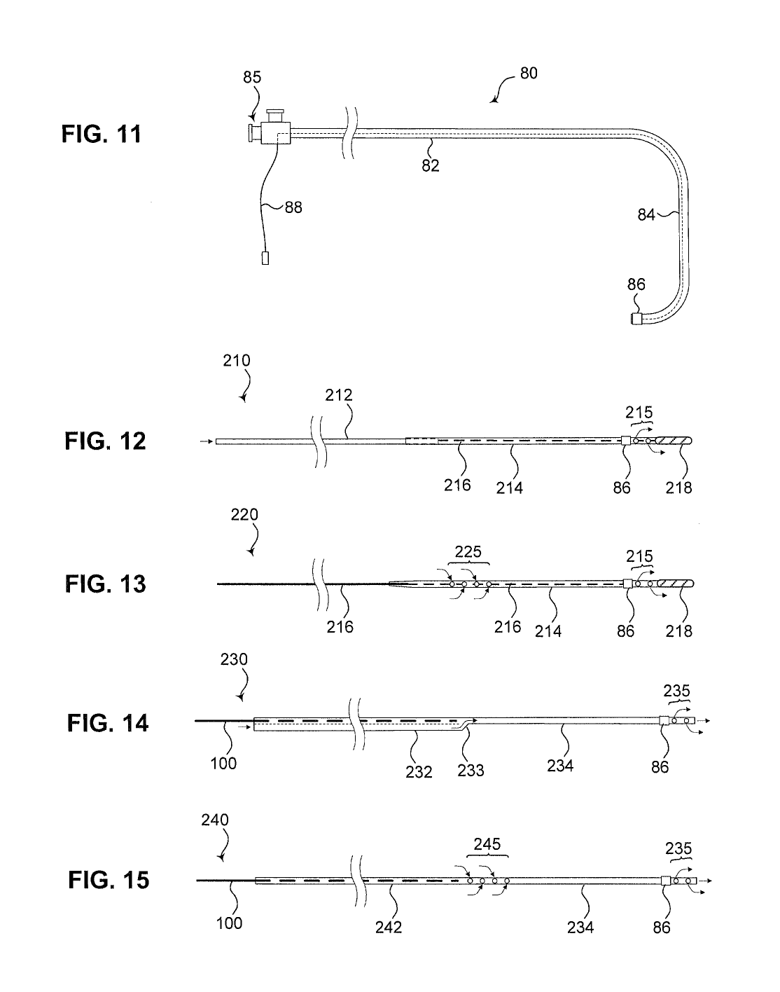

With reference to FIG. 11, an example configuration of guide catheter 80 is shown schematically. Guide catheter 80 may have a conventional construction including an elongate shaft 82 and a single lumen extending therethrough. The elongate shaft 82 may include a distal shaped section 84 to facilitate cannulation of the target coronary artery. The proximal end of the shaft 82 may be connected to a manifold 85 including a coaxial port and a side port, both in communication with the lumen extending through the shaft 82. Although not conventional, the guide catheter 80 may include one or more distal sensors 86 mounted adjacent the distal end of the shaft 82. The sensor 86 may comprise a temperature sensor (e.g., thermocouple), a pressure sensor (e.g., piezoelectric transducer), and/or a flow sensor (e.g., anemometer) as described previously. Electrical connection to the sensor 86 may be provided by insulated wires 88 embedded in the shaft 82 and proximally exiting the manifold 85. The wires 88 may be connected to the system 120 to enable feedback control as described previously.

With reference to FIG. 12, an example configuration of crossing device 90 in the form of an infusion guide wire 210 is shown schematically. The infusion guide wire 210 may include a proximal tubular shaft section 212 and a distal tubular shaft section 214. The proximal tubular shaft section 212 may comprise a relatively stiff tube (e.g., stainless steel hypotube or high durometer polymer) having an outside diameter equal to that of a conventional guide wire 100, e.g., 0.014 inches, such that conventional over-the-wire devices such as a balloon or stent delivery catheter may be advanced over the infusion guide wire 210. The distal tubular shaft section 214 may comprise a relatively flexible (e.g., lower durometer polymeric tube) having a similar outside diameter. A core wire 216 may be connected to and extend from the distal end of the proximal shaft section 212 and through the distal shaft section 214, terminating in a radiopaque coil 218 to form an atraumatic tip. One or more sensors 86 may be provided adjacent the distal end of the distal shaft section 214 with corresponding wires (not shown) extending proximally through both shaft sections 214 and 212. A series of one or more exit ports 215 may be provided in the distal tubular shaft section 214 adjacent the distal end thereof. Fluid may enter the proximal end of the proximal tubular shaft section 212, pass through the internal lumen, and exit distally of the restriction through side ports 215 as indicated by the arrows.

With reference to FIG. 13, another example configuration of crossing device 90 in the form of an alternative infusion guide wire 220 is shown schematically. Infusion guide wire 220 is similar to the infusion guide wire 210 described previously, except that fluid may enter ports 225 at the proximal end of the distal tubular shaft section 214. Thus, in this embodiment, cold fluid may be delivered via the guide catheter 80, pass down the coronary artery, enter side ports 225, and exit distally of the restriction through side ports 215. This configuration utilizes the lumen of the guide catheter 80 for the transport of fluid, negating the need for a tubular proximal shaft section on the infusion guide wire 220. Accordingly, the proximal end of the distal tubular shaft section 214 may be connected to and sealed about the core wire 216, which extends proximally therefrom to form the proximal shaft of the infusion guide wire 220.

With reference to FIG. 14, an example configuration of crossing device 90 in the form of an infusion catheter 230 is shown schematically. The infusion catheter 230 may include a proximal tubular shaft section 232 and a distal tubular shaft section 234. The proximal tubular shaft section 232 may comprise a flexible dual lumen polymeric tube (e.g., a single dual lumen extrusion or two coaxial single lumen extrusions) having an infusion lumen configured for the passage of fluid, and a guide wire lumen configured to accommodate a conventional guide wire 100. The distal tubular shaft section 234 may comprise a flexible single lumen polymeric tube defining a shared infusion and guide wire lumen. The shared lumen may be configured to accommodate advancement of the catheter 230 over a conventional guide wire 100, and configured to accommodate fluid delivery when the guide wire 100 is pulled back as shown. One or more sensors 86 may be provided adjacent the distal end of the distal shaft section 234 with corresponding wires (not shown) extending proximally through both shaft sections 234 and 232. A series of one or more exit ports 235 may be provided in the distal tubular shaft section 234 adjacent the distal end thereof. Fluid may enter the proximal end of the proximal tubular shaft section 232, pass through the proximal infusion lumen, pass through the distal shared lumen (when the guide wire is retracted proximal of the transition 233) and exit distally of the restriction through side ports 235 as indicated by the arrows.

With reference to FIG. 15, another example configuration of crossing device 90 in the form of an alternative infusion catheter 240 is shown schematically. Infusion catheter 240 is similar to the infusion catheter 230 described previously, except that fluid may enter ports 245 at the proximal end of the distal tubular shaft section 234. Thus, in this embodiment, cold fluid may be delivered via the guide catheter 80, pass down the coronary artery, enter side ports 245 (when the guide wire 100 is retracted proximal of the entry ports 245), and exit distally of the restriction through side ports 235. This configuration utilizes the lumen of the guide catheter 80 for the transport of fluid, negating the need for a dual lumen tubular proximal shaft section. Accordingly, the proximal shaft section 242 may be a continuation of the single lumen distal tubular shaft section 234 extending proximally.

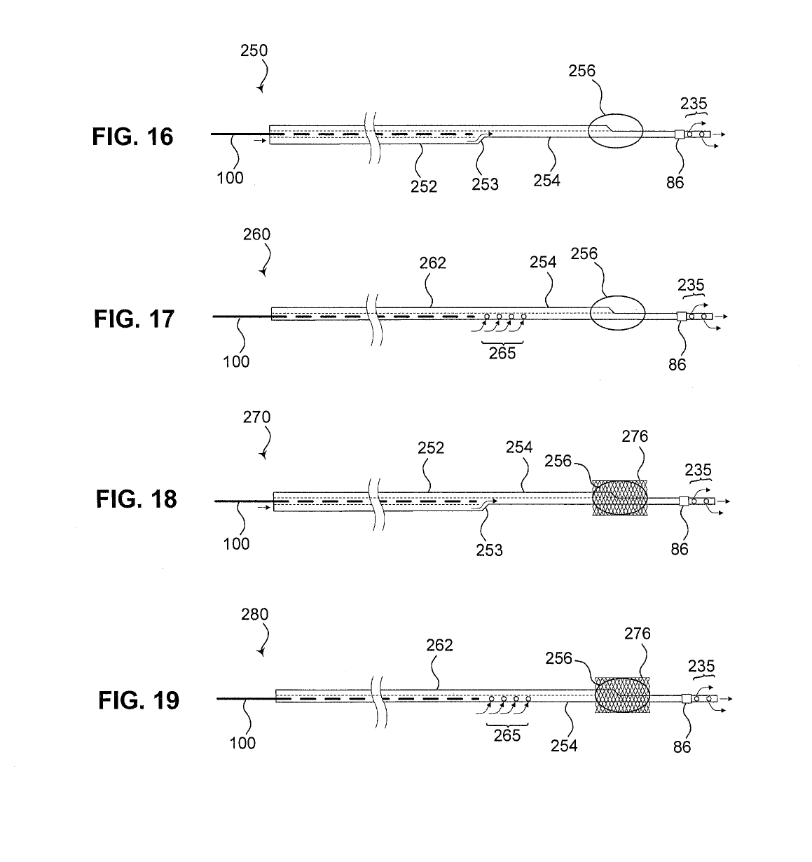

With reference to FIG. 16, an example configuration of crossing device 90 in the form of a balloon catheter 250 is shown schematically. The balloon catheter 250 may include a proximal tubular shaft section 252 and a distal tubular shaft section 254. The proximal tubular shaft section 252 may comprise a flexible triple lumen polymeric tube (e.g., a single triple lumen extrusion) having an inflation/deflation lumen configured for inflating and deflating the balloon 256, an infusion lumen configured for the passage of fluid, and a guide wire lumen configured to accommodate a conventional guide wire 100. The distal tubular shaft section 254 may comprise a flexible dual lumen polymeric tube defining an inflation/deflation lumen and a shared infusion and guide wire lumen. The shared lumen may be configured to accommodate advancement of the catheter 250 over a conventional guide wire 100, and configured to accommodate fluid delivery when the guide wire 100 is pulled back proximal of the transition 253 as shown. The balloon 256 may be used to dilate the culprit restriction. One or more sensors 86 may be provided adjacent the distal end of the distal shaft section 254 with corresponding wires (not shown) extending proximally through both shaft sections 254 and 252. A series of one or more exit ports 235 may be provided in the distal tubular shaft section 254 distal of the balloon 256. Fluid may enter the proximal end of the proximal tubular shaft section 252, pass through the proximal infusion lumen, pass through the distal shared lumen (when the guide wire is retracted proximal of the transition 253) and exit distally of the balloon 256 and restriction through side ports 235 as indicated by the arrows. Thus, the infusion lumen may be used to delivery cold fluid for pre-conditioning before and during balloon 256 inflation, controlling reperfusion dynamics before and during balloon 256 deflation, and post-conditioning after balloon 256 deflation.

With reference to FIG. 17, another example configuration of crossing device 90 in the form of an alternative balloon catheter 260 is shown schematically. Balloon catheter 260 is similar to the balloon catheter 250 described previously, except that fluid may enter ports 265 at the proximal end of the distal tubular shaft section 254. Thus, in this embodiment, cold fluid may be delivered via the guide catheter 80, pass down the coronary artery, enter side ports 265 (when the guide wire 100 is retracted proximal of the entry ports 265), and exit distally of the balloon 256 and restriction through side ports 235. This configuration utilizes the lumen of the guide catheter 80 for the transport of fluid, negating the need for a triple lumen tubular proximal shaft section. Accordingly, the proximal shaft section 262 may be a continuation of the dual lumen distal tubular shaft section 254 extending proximally.

With reference to FIGS. 18 and 19, each schematically show an example configuration of crossing device 90 in the form of a stent delivery catheter 270 and 280, respectively. The configuration of stent delivery catheters 270 and 280 is the same as balloon catheters 250 and 260, respectively, except for the provision of a stent 276 on the balloon 256. Similar to balloon catheters 250 and 260, the infusion lumens in the stent delivery catheters 270 and 280 may be used for the delivery cold fluid for pre-conditioning before and during stent 276 deployment, controlling reperfusion dynamics during and after balloon 256 deflation, and post-conditioning after balloon 256 deflation.

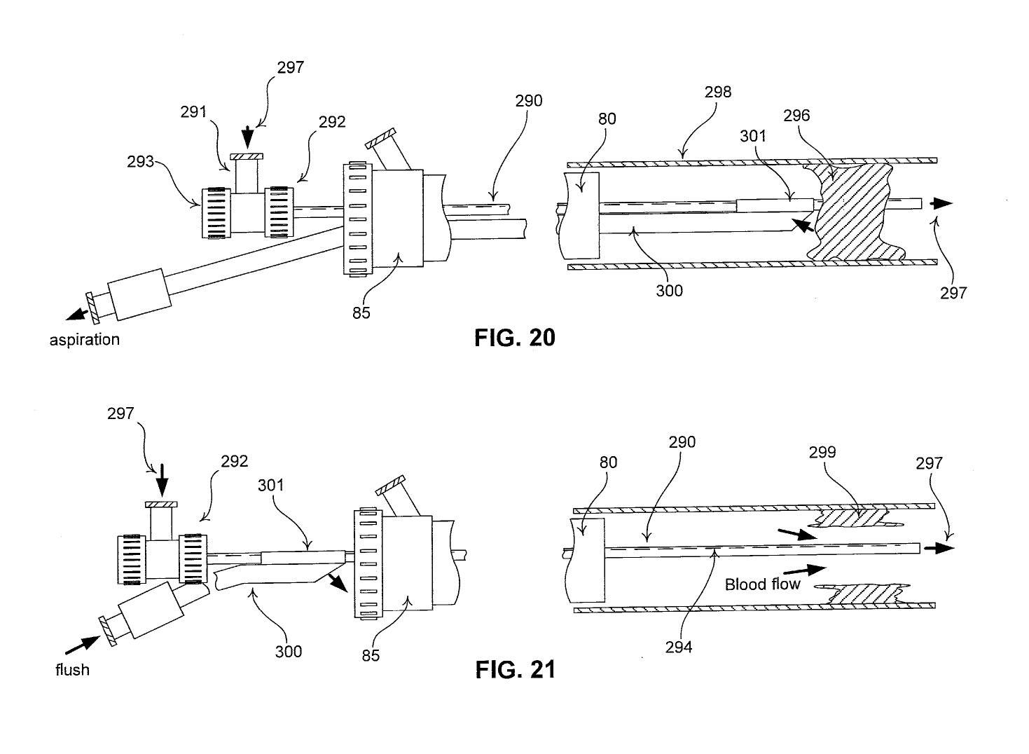

FIGS. 20-50 schematically illustrate various embodiments of devices that may be used in an antegrade hypothermia system. Devices and features described with reference to a device in one embodiment may be applied to other embodiments.

With reference to FIGS. 20 and 21, an alternative embodiment is shown schematically, where crossing device 90 is in the form of an infusion catheter 290, which is configured to deliver chilled fluid via an internal lumen (not shown). Infusion catheter 290 may be delivered over a conventional guide wire 100, which is typically about 0.014 inch diameter. Internal lumen may be about 0.016 inch inner diameter to accommodate the guide wire. Some or all of the internal lumen may alternatively be larger, say from 0.016 to 0.025 inch to provide for lower resistance to fluid flow. It is desirable to utilize a thin wall, to minimize the outer diameter dimension. A small outer diameter will tend to minimize the initial disruption to the blockage, and minimize reperfusion of blood prior to the distal tissue being cooled. In the case of a uniform inner diameter of about 0.016 inch, the wall thickness is preferably about 0.001 to 0.003 inch. By having a minimal outer diameter, a thrombus removal catheter 300 may be advanced over the outside of the infusion catheter 290. The thrombus removal catheter 300 may comprise an aspiration catheter or a thrombectomy catheter, for example. The tracking lumen 301 of the thrombus removal catheter 300 may be monorail or rapid exchange configuration, as illustrated. The thrombus removal catheter 300 further includes an aspiration lumen (not shown) which extends through the catheter. Infusion catheter 290 may include one or more side holes (not shown) near the distal tip for minimizing jetting of fluid from the distal opening.

In this embodiment, the thrombus removal catheter 300 may be advanced directly over the infusion catheter 290. With this arrangement, infusion of cooled fluid 297 may be continued during the thrombus removal step. Initially, the artery may be cannulated with a guide catheter 80. The guide catheter may include a conventional Y-adaptor (manifold) 85, as is known in the art. A guide wire 100 (not shown) may be used to initially cross the blockage 296. The infusion catheter 290 may then be advanced through the blockage, and the guide wire removed. Infusion of cooled solution 297 may then start to pre-cool the ischemic tissue prior to any substantial reperfusion via an infusion port 291.

The thrombus removal catheter 300 may then be advanced down the outer diameter of the infusion catheter 290. The proximal end of infusion catheter 290 may include a removable hub 292 to facilitate placement of the tracking lumen 301 of the thrombus removal catheter 300 over the proximal end of the infusion catheter 290. Alternatively, thrombus removal catheter may be “pre-loaded” onto the infusion catheter 290 prior to the insertion of the infusion catheter 290 in the patient. The hub of the infusion catheter 290 may further include an adjustable seal 293 on the proximal end for sealing around the guide wire, or sealing off the guide wire access port.

Infusion down the infusion catheter 290 may be continued during the aspiration of the thrombus. In this manner, the distal ischemic myocardium is maintained in a cooled state upon exposure to the normal blood flow upon removal of the thrombus.

If the thrombus removal catheter 300 becomes clogged, it can be retracted out of the guide catheter, as shown in FIG. 21, flushed, and reinserted into the patient. The whole time, infusion of cooled solution may be maintained.

After the thrombus is removed, the thrombus removal catheter 300 may be removed from the infusion catheter 290. The optional removable hub 292 may facilitate this by first removing the hub, then the thrombus removal catheter. Infusion through the infusion catheter 290 may be re-established if desired, while a guide wire (not shown) is advanced alongside the infusion catheter 290. A stent delivery catheter (not shown) may then be used to place a stent in the underlying lesion, while infusion of cooled solution is maintained. Preferably, prior to the stent being deployed, the infusion catheter 290 is withdrawn proximally of the lesion to avoid interfering with the stent expansion. In the manner described in this paragraph, infusion of cooled solution is substantially maintained during the stent placement step. Infusion of cooled fluid may also be continued after the stent placement. For this embodiment and all others, the infusion rate following the thrombus removal and or following the stent placement may be increased compared to the flow rate following the initial crossing of the blockage 296, to factor in the competing antegrade flow of blood upon the opening up of the blockage 296. This can be facilitated by simply increasing the flow through the infusion catheter 290, if it is in place at the desired time, or if the thrombus removal catheter 300 is in place at the desired time (e.g. following thrombus removal, the thrombus removal catheter 300, which typically has a relatively large lumen, may already be in place) infusion through it may be performed.

Alternatively, following removal of the thrombus removal catheter 300, the lumen of the infusion catheter 290 may be used to place the guide wire 100 back across the blockage 296. Infusion of cooled solution 297 would likely be stopped during this step. Once the guide wire is across the lesion 299, the infusion catheter 290 is removed from the guide wire. In this manner, the initial access across the blockage 296 is maintained throughout the procedure. The infusion catheter 290 may include a peel-away feature 294 along all or most of its length to facilitate such removal, if the guide wire is not an exchange length guide wire. Peel away feature 294 may be a thin or weakened portion of the catheter wall, such as partial slice through the thickness of the wall. A stent delivery catheter 310 (not shown here) may be advanced down the guide wire 100 to stent the residual lesion 299.

Cooling of the tissue may be continued (not shown) via reintroduction of the thrombus removal catheter 300 (or a conventional infusion catheter), advanced over the guide wire 100 following removal of the stent delivery catheter 310.

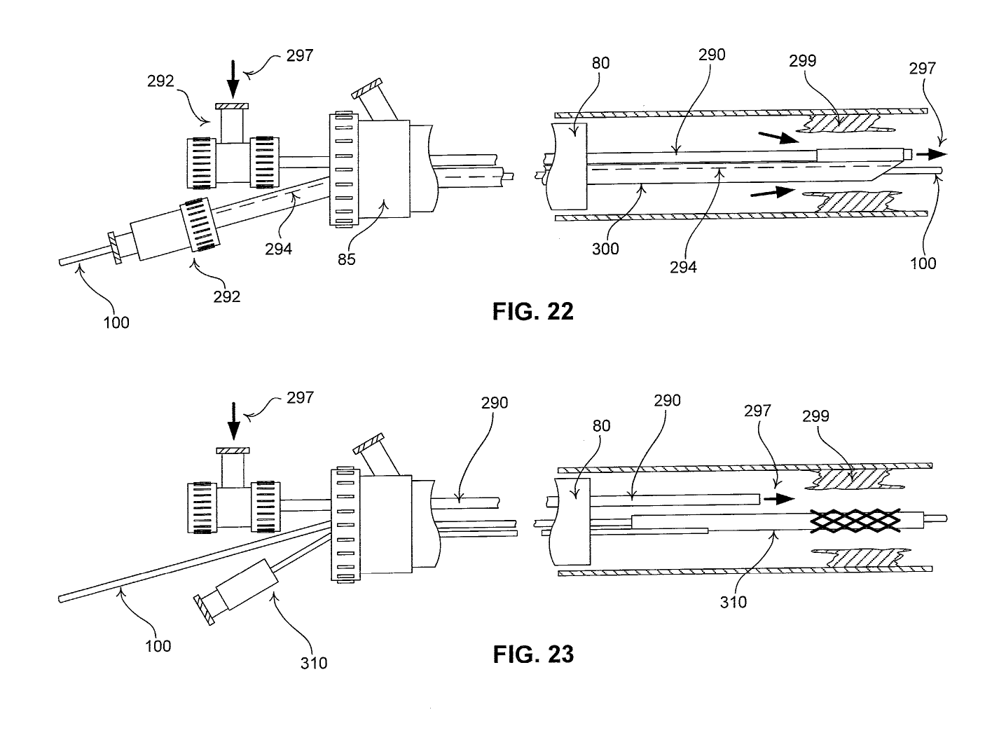

With reference to FIGS. 22 and 23, an alternative embodiment is shown in which the thrombus removal catheter 300 incorporates a removable hub 292 and a peel-away feature 294. In use, infusion catheter 290 of this embodiment may be placed initially across the blockage 296 with a conventional guide wire 100, which may then be removed to facilitate infusion of cooled fluid 297. Optionally, the infusion catheter 290 may have a rapid exchange configuration as shown and described with reference to FIG. 37. Aspiration of the thrombus 296 may be performed with a thrombus removal catheter 300 that may be advanced directly over the infusion catheter 290.

After the thrombus 296 is removed, the thrombus removal catheter 300 is positioned across the residual lesion 299. A guide wire 100 is then advanced down the aspiration lumen of the thrombus removal catheter 300, re-establishing the guide wire position distal of the lesion 299, as illustrated in FIG. 22 (for subsequent stent placement). The thrombus removal catheter 300 may then be removed from the guide wire 100, by removal of the removable hub 292, and then by peeling off the catheter from the guide wire 100 utilizing the peel-away feature 294. A stent delivery catheter 310 may then be positioned across the residual lesion 299, as shown in FIG. 23 and a stent delivered. Infusion of cooled fluid 297 may continue through the infusion catheter 290, substantially uninterrupted, during all the steps described in this paragraph. Cooling may continue after the stent has been placed. Furthermore, this embodiment provides for maintaining an access across the lesion for the entire procedure following the initial placement of the guide wire 100.

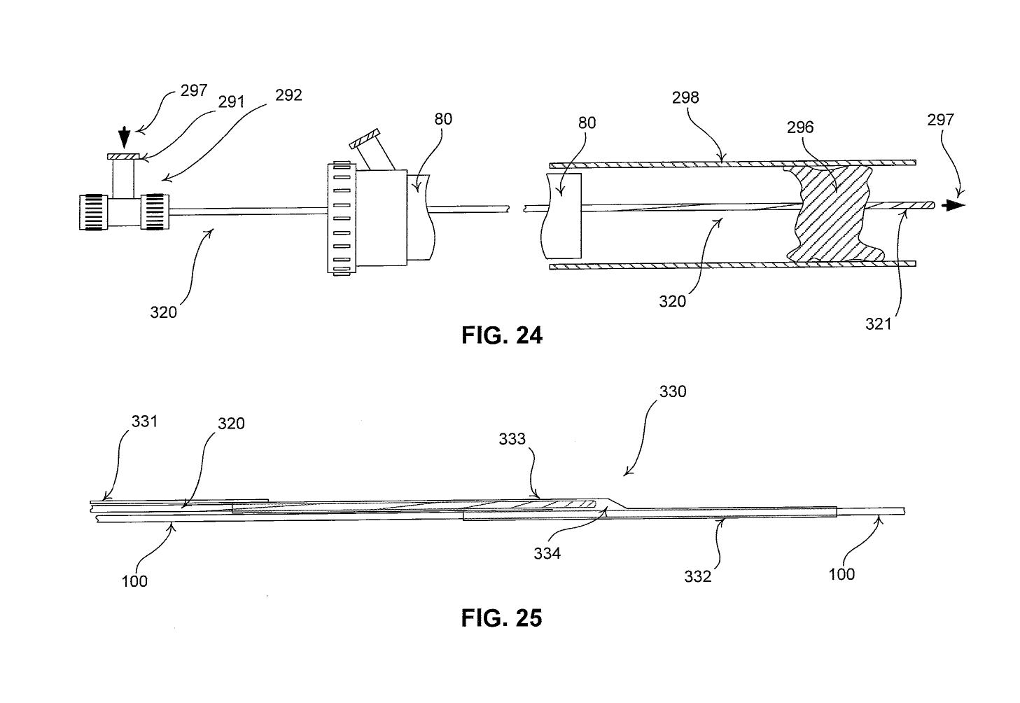

With reference to FIGS. 24-27, alternative embodiments are schematically illustrated wherein an infusion guide wire 320 may be used to initially cross the blockage and initiate infusion of chilled fluid 297. Infusion wire 320 has an internal lumen (not shown) for infusion via an infusion port 291. The hub 292 may be removable, to allow for other devices to be advanced down it, as will be described below. The infusion wire 320 may perform like conventional guide wires, exhibiting steerability, trackability and other characteristics common to conventional guide wires. It may be fabricated from a metallic hollow tube, such as from stainless steel or nickel titanium alloy. A spiral cut pattern 321 near the distal end may provide for added flexibility near the distal end. The cold fluid 297 may flow from a hollow open tip, or from the spiral cut, or both. A thermal insulating layer (not shown) may cover the exterior to minimize heat exchange through the wall of the infusion wire 320. The outer diameter of the infusion wire 320 may be about 0.014 inch, to facilitate its use with other typical coronary catheters.

In use, the infusion guide wire 320 may be placed through a guide catheter 80, across the blockage 296 in the blood vessel 298. Then infusion of cooled fluid 297 may begin, which cools the distal ischemic myocardium prior to substantial reperfusion with blood. As the infusion wire 320 may be dimensionally compatible with a multitude of conventional coronary devices, the infusion of cooled fluid may continue, substantially uninterrupted, during thrombus removal, stent delivery, or other desired procedures that may be performed over a guide wire. Interruption of cooled fluid infusion would only be necessary during removal and reinstallation of the hub 292, when other catheters, e.g. thrombectomy or stent catheters, are loaded onto the infusion wire 320.

Although it is desired to use the infusion wire 320 as a guide wire, in some instances it may not perform as well as a conventional guide wire 100 for accessing and crossing the blockage 296. In this case, as seen in FIG. 25, a wire exchange sleeve 330, together with a conventional guide wire 100 may be used for initial crossing.

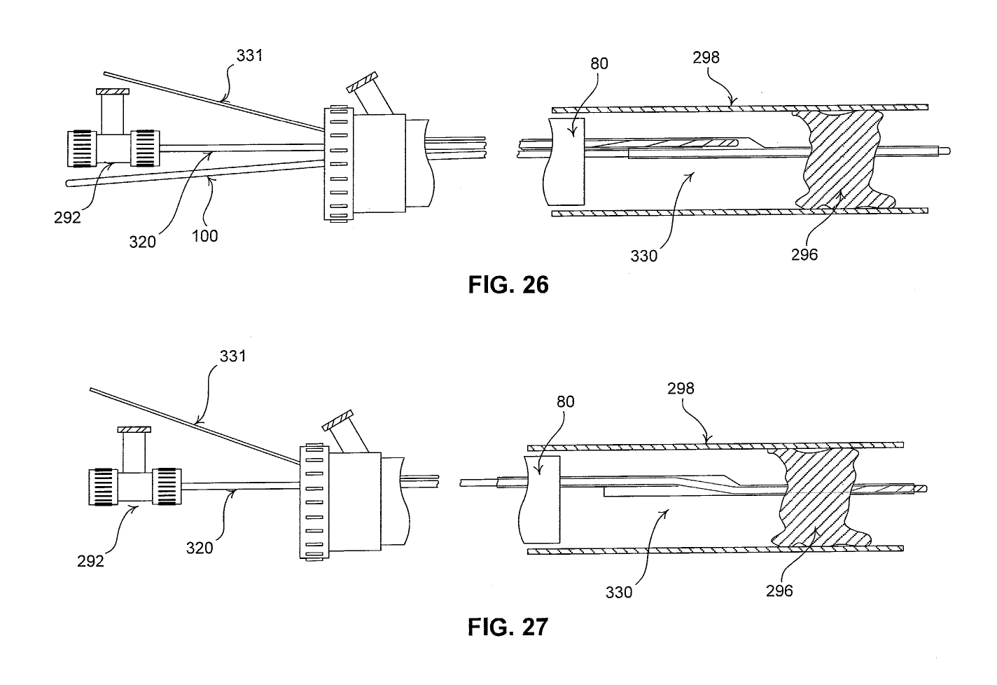

Wire exchange sleeve includes a shaft 331, a wire lumen 332, a parking lumen 333, and an opening 334 between the wire lumen and the parking lumen. In use, a conventional guide wire 100 is used to cross the blockage 296. Then the exchange sleeve 330, together with the infusion wire 320 placed in the parking lumen 333 is loaded over the guide wire 100 via the wire lumen 332. The exchange sleeve 330 and infusion wire 320 may be advanced down the guide wire 100 until the tip of the exchange sleeve 330 is across the blockage 296, as seen in FIG. 26. Then the guide wire 100 may be removed, and the infusion wire 320 advanced from the parking lumen 333 into and through the wire lumen 332 and across the blockage 296, as seen in FIG. 27. At this point, the wire exchange sleeve 330 may be removed, and the infusion of cooled solution begun.

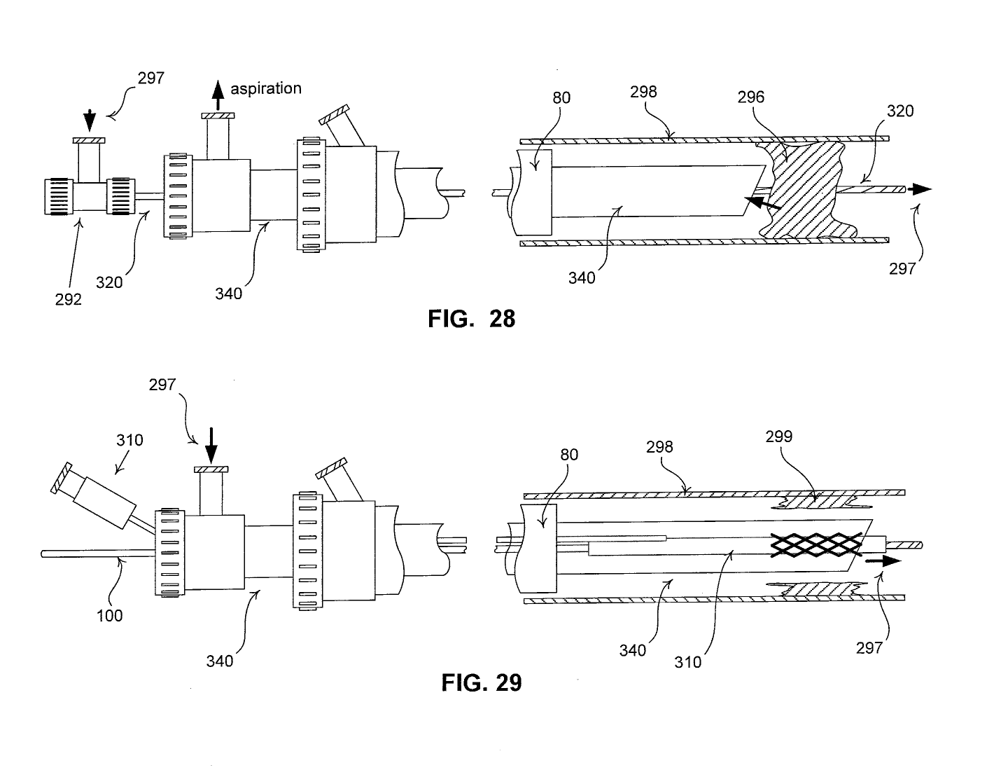

With reference to FIGS. 28 and 29, a further embodiment is shown schematically wherein then infusion wire 320 is positioned within an aspiration sheath 340. The aspiration sheath 340 may be positioned within a conventional guide catheter 80 to access the blood vessel of interest. The infusion wire 320 may be placed across the blockage 296 with the aid of an wire exchange sleeve 330 as described in the embodiment of FIG. 25.

Once the infusion wire 320 is across the blockage 296, infusion of cooled fluid 297 may be initiated to the ischemic tissue distal of the blockage 296. Thrombus may be then removed by utilizing the aspiration sheath 340 as a thrombectomy catheter, as shown in FIG. 28. Cooling may be continued via the infusion wire 320 during this step. Once the thrombus is removed, it may be desirable to remove the infusion wire 320. Thrombus may “hang up” within the lumen of the aspiration sheath 340, but if the infusion wire 320 (or any device in the lumen) is removed while suctioning, any thrombus particulate will be removed. This can be important if the thrombectomy (aspiration) catheter is used subsequently for infusion.

Next, while the aspiration sheath 340 is positioned distally of the residual lesion 299, a conventional guide wire 100 may be placed distally, thus preserving access for subsequent stent placement. Infusion of cooled fluid may be performed during this and following steps.

A stent delivery catheter 310 may then be advanced along the guide wire 100, through the lumen of the aspiration sheath 340 and across the residual lesion 299, as shown in FIG. 29. Infusion of cooled fluid 297 may be continued during this step. The aspiration sheath 340 is then retracted to a position proximal of the lesion 299 and the stent delivered. Infusion of cooled fluid can be continued following the delivery of the stent.

With this embodiment, as well as others describe here, the commonly performed functions of contrast delivery and aortic pressure monitoring normally done via the guide catheter may be performed all or in part via the aspiration sheath 340 (or thrombectomy catheters 300 in other embodiments), as these catheters will possess the relatively large lumen needed for these functions. Small amounts of contrast delivery may also be delivered via the infusion catheters of the various embodiments described here.

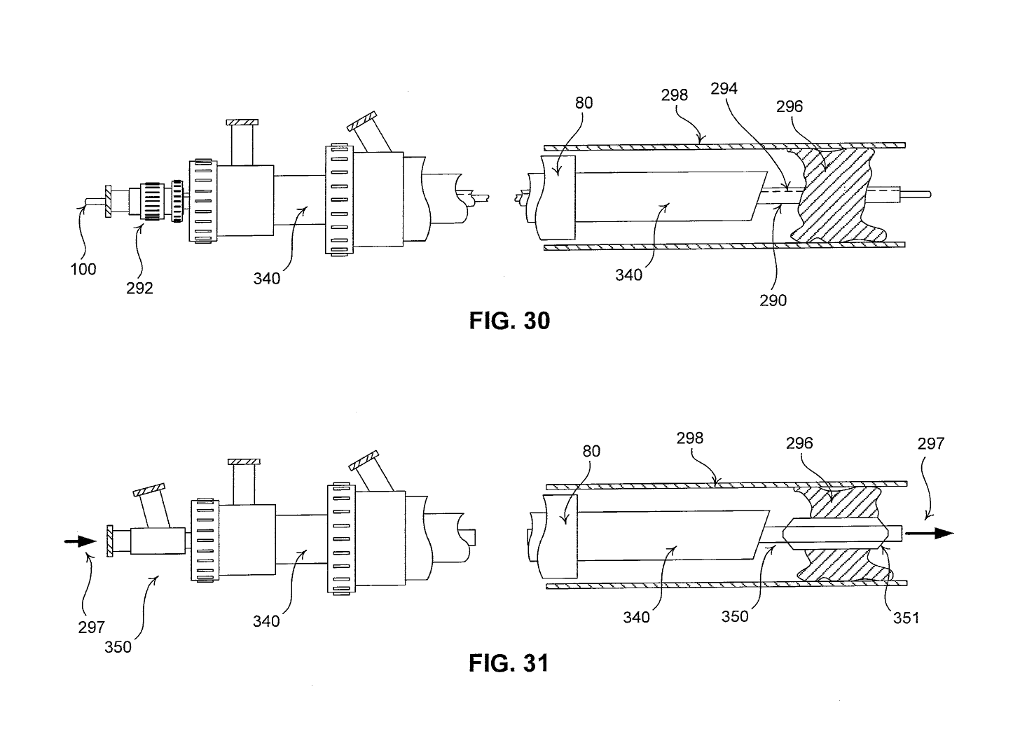

With reference to FIG. 30, an alternative embodiment is shown schematically wherein a small diameter infusion catheter 290, positioned with the aid of a conventional guide wire 100, may be utilized for the initial cooling step. The guide wire 100 may be removed to facilitate the infusion of cooled fluid 297 through the lumen of the infusion catheter 290. Infusion catheter 290 may have a removable hub 292 and further may have a peel-away feature 294, but as described below, these features may not be necessary.

In this embodiment, the aspiration sheath 340 may be utilized for removing thrombus. Infusion may continue through the infusion catheter 290. Once the thrombus is removed, the infusion catheter 290 and guide wire 100 are preferably removed (during suction) to assure no thrombus remains in the aspiration lumen. At this time, the guide wire 100 can be re-positioned across the residual lesion 299 via the aspiration lumen of the aspiration sheath 340. Infusion of cooled fluid 297 can be re-instated. A stent delivery catheter 310 may be advanced on the guide wire 100 and through the aspiration lumen to the lesion 299 and the lesion 299 stented. Infusion of cooled fluid may continue following the stent delivery.

With reference to FIG. 31, an alternative embodiment is schematically illustrated wherein the initial crossing of the blockage 296 is performed with a balloon catheter 350, which may incorporate a relatively small diameter dilation balloon 351 (e.g. a “pre-dilation” catheter). The blood vessel 298 may be accessed with a guide catheter 80. An aspiration sheath 340 is positioned within the guide catheter 80. A guide wire 100 (not shown) may be used with the balloon catheter 350 to facilitate navigation to and crossing of the blockage 296. Once the balloon 351 is across the blockage 296, it is inflated. Such inflation within the blockage 296 is believed to further assure against any antegrade flow of blood prior to the infusion of cooled fluid. The guide wire 100 may then be removed and infusion begun via the guide wire lumen in the balloon catheter 350. This is the stage illustrated in FIG. 31. Balloon catheter 350 is preferably an “over the wire” type, having a lumen extending longitudinally therethrough.

The aspiration sheath 340 may then be used to aspirate the thrombus, while distal infusion of cooled fluid 297 may continue. Once the thrombus is aspirated, the balloon catheter 350 is preferably withdrawn (during suction via the thrombus removal catheter) to assure complete removal of thrombus from the aspiration lumen. A guide wire 100 may then be positioned through the lumen of the aspiration sheath 340 and across the residual lesion 299. Cooling may be re-instated via the aspiration lumen. A stent delivery catheter 310 may be positioned through the lumen of the aspiration sheath 340 and across the blockage 296, and delivered. Cooling may be continued via the aspiration sheath 340 following stent delivery.

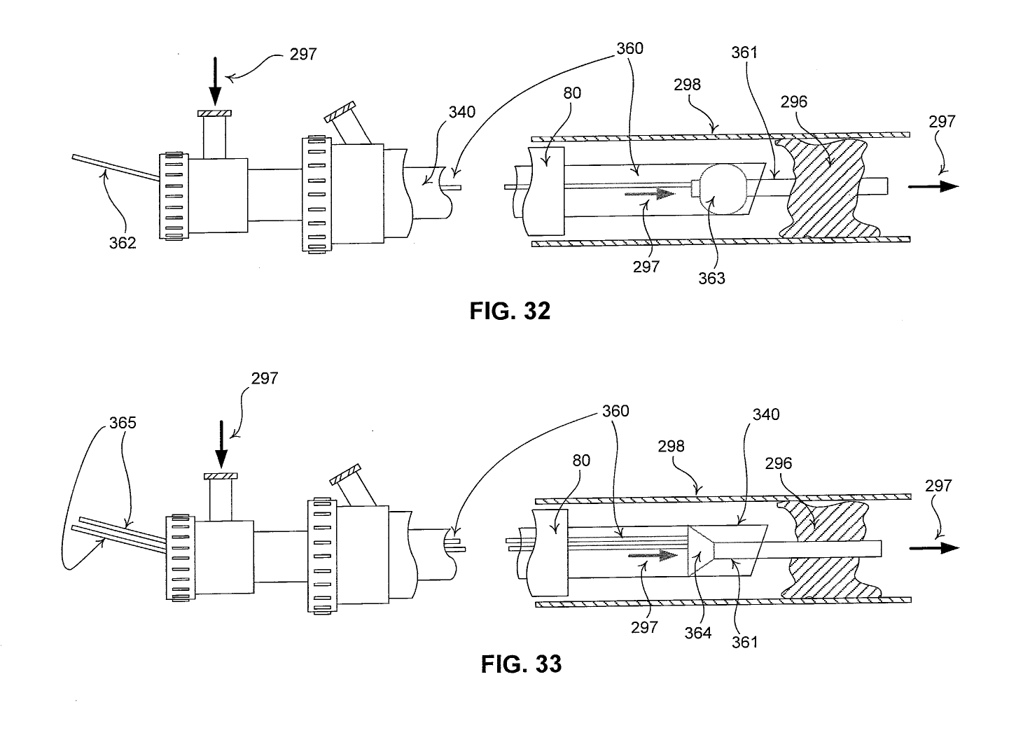

With reference to FIG. 32, an alternative embodiment is schematically shown wherein the infusion catheter 360 makes use of the lumen of the thrombus removal catheter 340 during infusion, rather than incorporating a full length infusion lumen. This may simplify the connections on the proximal end, as the aspiration port 341 on the thrombus removal catheter 340 also serves to be the infusion port during the times during the procedure where infusion of cooled fluid 297 is desired.

Infusion catheter 360 includes a distal shaft portion 361, a proximal shaft portion 362, and a sealing balloon 363, positioned near the proximal end of the distal shaft portion 361. Note that the thrombus removal catheter 340 is shown semi-transparent at the distal end. Distal shaft portion 361 includes an infusion lumen (not shown), extending from its proximal end to the distal tip. Proximal shaft portion 362 includes an inflation lumen (not shown) which facilitates inflation of the sealing balloon 363. Sealing balloon 363 serves to seal the distal shaft portion 361 to the interior of the thrombus removal catheter 340. Thus when cooled solution 297 is conveyed down the lumen of the thrombus removal catheter 340, it is directed through the infusion lumen and out the distal tip.

In use, the infusion catheter 360 and thrombus removal catheter 340 may be positioned within a guide catheter 80, as shown. A guide wire 100 (not shown) may further be utilized within the infusion catheter 360 to facilitate initial crossing of the blockage 296. The guide wire 100 may be removed. Cooled fluid 297 may be infused through the thrombus removal catheter 340 and the infusion catheter 360 to cool the distal tissue prior to any substantial reperfusion of blood.

Aspiration of thrombus may then be performed by removing the infusion catheter 360, and then advancing the thrombus removal catheter 340 into the thrombus while aspirating. Alternatively, the thrombus removal catheter 340 may be left in place, the sealing balloon 363 deflated, and the thrombus removal catheter 340 advanced over the infusion catheter 360 while aspirating. Following thrombus removal, the thrombus removal catheter 340 may be temporarily retracted to assure complete thrombus removal from the aspiration lumen. After the thrombus is removed, a guide wire 100 may be re-positioned across the residual lesion 299 by using the infusion lumen of the infusion catheter 360. The sealing balloon 363 may be temporarily re-inflated to help direct the tip of the guide wire 100 into the infusion lumen and into the distal blood vessel.

Once the guide wire 100 is across the residual lesion 299, the infusion catheter 360 may be removed, and a stent delivery catheter 310 advanced over the guide wire 100, through the thrombus removal catheter 340, and across the lesion 299 to deliver a stent. Cooled fluid 297 may be delivered following the stent delivery by infusing through the thrombus removal catheter 340.

FIG. 33 illustrates an alternative embodiment, similar to that described in FIG. 32. An expandable and retractable funnel 364 (rather than a sealing balloon) may be incorporated on the proximal end of the distal portion 361 of the infusion catheter 360. The proximal shaft 362 of the infusion catheter 360 comprises one or more actuation elements 365 that serve to expand and retract the funnel 364. Funnel 364 may comprise a braid structure that expands upon foreshortening imparted by relative axial motion between two actuation elements 365.

In use, this embodiment is also similar to that described in connection with FIG. 32, but funnel 364 expansion and retraction replaces the balloon 363 inflation and deflation steps. The funnel 364 also serves to easily direct a guide wire 100 back into the infusion lumen at times when a guide wire 100 may be repositioned therein.

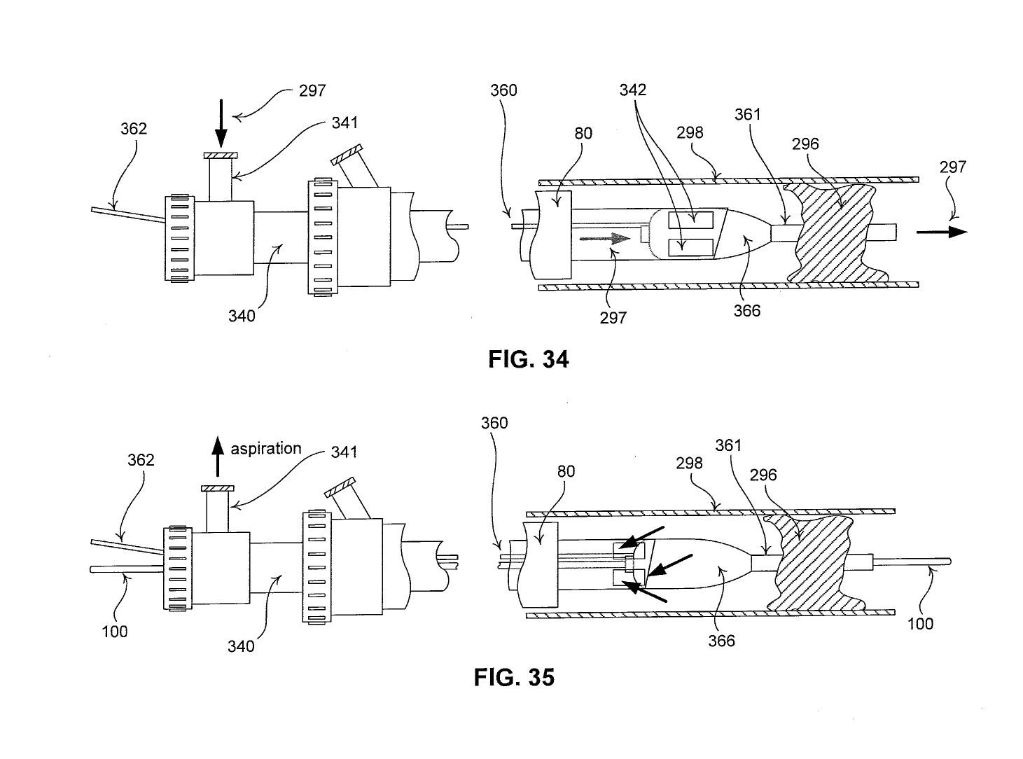

FIGS. 34-36 show an alternative embodiment of an infusion and thrombus removal catheter system. System may include an thrombus removal catheter 340 and an infusion catheter 360. Thrombus removal catheter 340 may include aspiration windows 342. Infusion catheter 360 may include an obturator 366, which may be solid, or inflatable, near the proximal end of the distal portion 361. Infusion catheter further includes an infusion lumen (not shown) extending through the distal portion 361, and a proximal portion 362, which may be an elongate shaft. Note that thrombus removal catheter 340 is shown semi-transparent in the distal region.

In use, the infusion catheter 360 and thrombus removal catheter 340 may be advanced into a blood vessel 298 with a guide catheter 80. Aspiration windows 342 are initially blocked by the obturator 366, as shown. A guide wire 100 (not shown) may also be used to facilitate advancement of the distal portion 361 of the infusion catheter 360 across a blockage 296. Following guide wire 100 removal, infusion of cooled fluid 297 may be initiated. Fluid is conveyed to the port 341, down the aspiration lumen and into the infusion lumen, and distally of the blockage 296.

Thrombus at the blockage 296 may be removed by first retracting the thrombus removal catheter 340 until the aspiration windows 342 are uncovered. Aspiration via the port 341 allows thrombus to be aspirated, as seen in FIG. 35, when the thrombus removal catheter 340 and infusion catheter 360 are advanced, preferably together, across the blockage 296. After the thrombus is removed, the infusion catheter 360 is removed. The obturator 366 serves to help clear all thrombus from the lumen of the thrombus removal catheter 340.

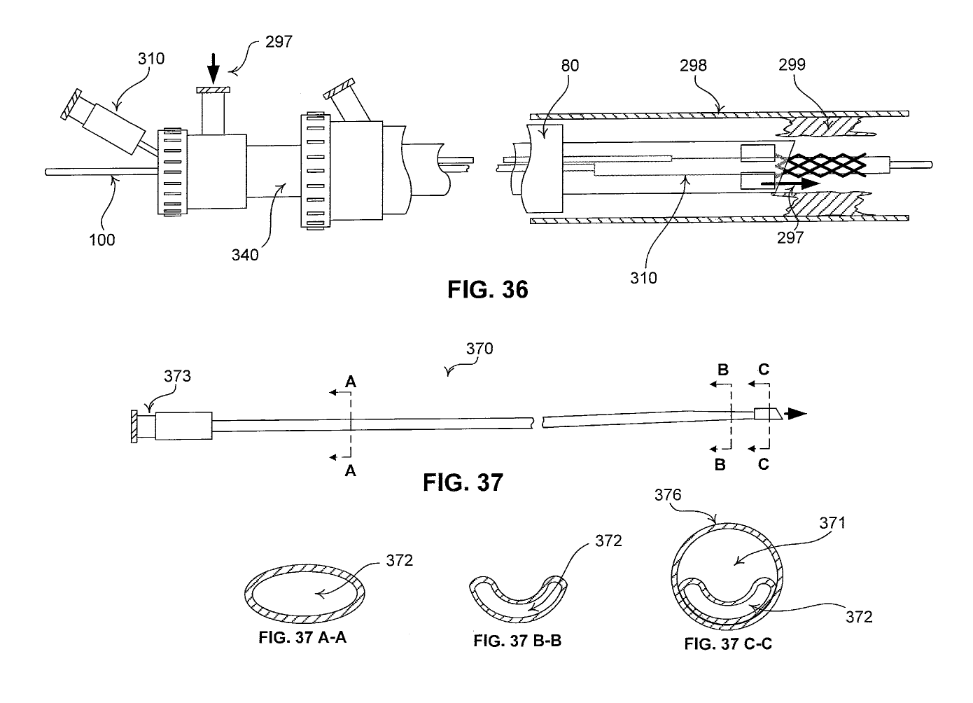

A stent delivery catheter 310 may then be advanced on the guide wire 100, through the thrombus removal catheter 340, as seen in FIG. 36. Following stent delivery to the residual lesion 299, infusion of cooled fluid 297 may be re-instated down the thrombus removal catheter 340.

FIGS. 37-41 show an alternative embodiment of an infusion and thrombus removal catheter system. Infusion catheter 370 may include a relatively short rail lumen 371 near the distal end. Infusion catheter 370 also includes an infusion lumen 372 that extends essentially the full length of the catheter, with an infusion port 373 at the proximal end. The infusion lumen 372 at the tip may have a non-circular cross section. Cross-sectional views taken along lines A-A, B-B and C-C in FIG. 37 are shown in FIGS. 37A-A, 37B-B and 37C-C, respectively. The infusion catheter 370 is at times next to an thrombus removal catheter 300 or a stent delivery catheter 310. As such, to minimize the diametric space it occupies within the constraints of the guide catheter 80, some or all of the length of the catheter 370 may be non-circular, as shown in FIG. 37A-A.

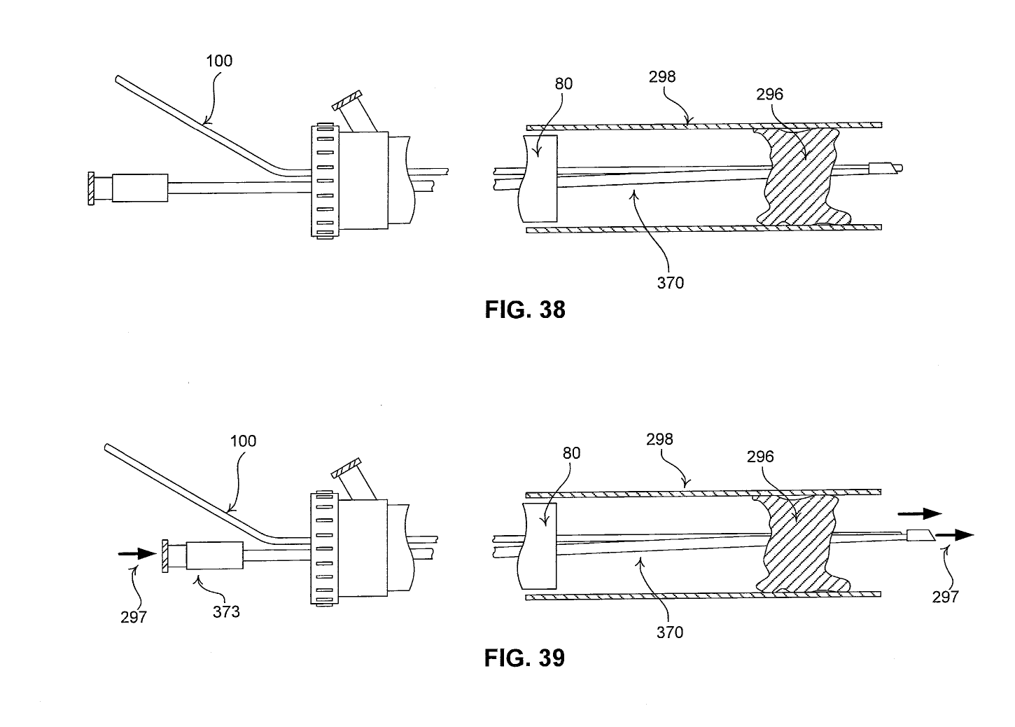

In use, the infusion catheter 370 may be advanced down a guide wire 100, and through a guide catheter 80, as seen in FIG. 38, until the tip of the infusion catheter 370 is positioned distal of the blockage 296. Radiographic contrast may be delivered down the infusion lumen 372 to aid in confirming that the tip is beyond the blockage 296.

Alternatively, infusion catheter 370 may be formed of a single lumen but have a sideport along a point near the distal end of the shaft. The segment between the distal tip and the sideport then forms the relatively short rail lumen described above.

Once the tip is beyond the blockage 296, the infusion catheter 370 may be further advanced (or the guide wire retracted, or both) until the rail lumen 371 is distal of the distal tip, as seen in FIG. 39. Infusion of cooled fluid 297 may be performed by infusing down the infusion lumen 372.

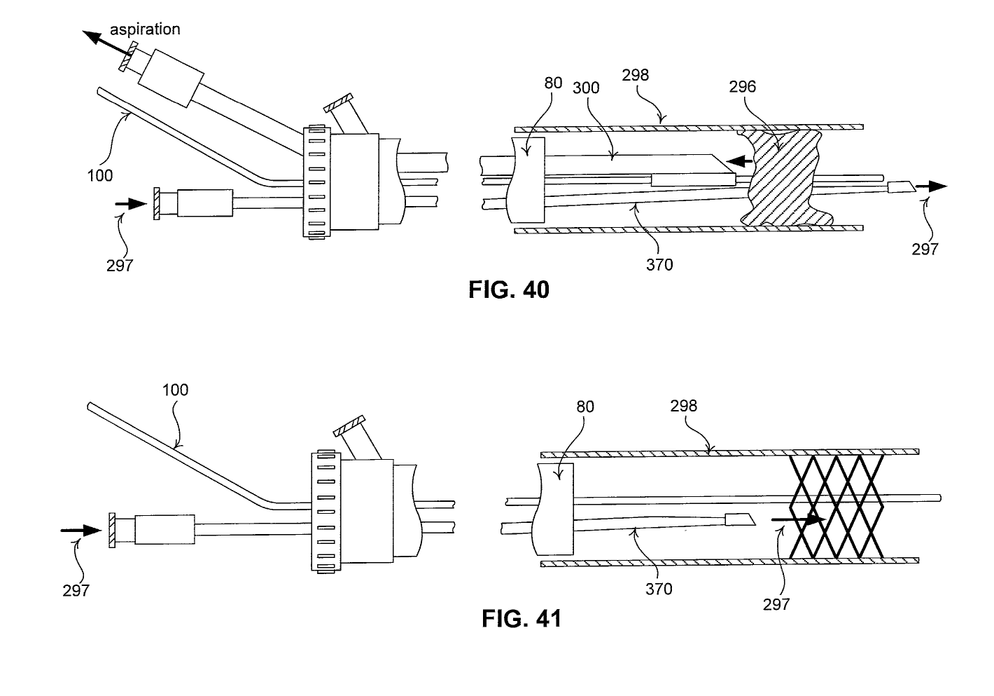

As the guide wire 100 is now fully separated, but still across the blockage 296, it can be used to guide the placement of an aspiration (thrombectomy) catheter 300, as seen in FIG. 40. Thrombus removal catheter 300 may be a monorail style catheter, as seen in the figure, or may be a full length over the wire catheter, if used with a long enough guide wire (e.g. an “exchange length” guide wire). Thrombus removal catheter 300 may be used to aspirate the thrombus. Infusion of cooled fluid 297 may continue through the infusion catheter 370 during the thrombus removal step.

After thrombus is removed, the aspiration/thrombus removal catheter 300 may be removed from the guide wire 100. A stent delivery catheter 310 may be advanced down the guide wire 100 and across the residual lesion 299. The stent may be delivered, and infusion of cooled fluid 297 may be continued, either down the infusion catheter 370, as shown in FIG. 41, or down another catheter, such as the thrombus removal catheter 300. In some situations, it may be advantageous to infuse cooled fluid 297 at a relatively high flow rate following placement of the stent. Therefore a catheter with a relatively large lumen may be desirable.

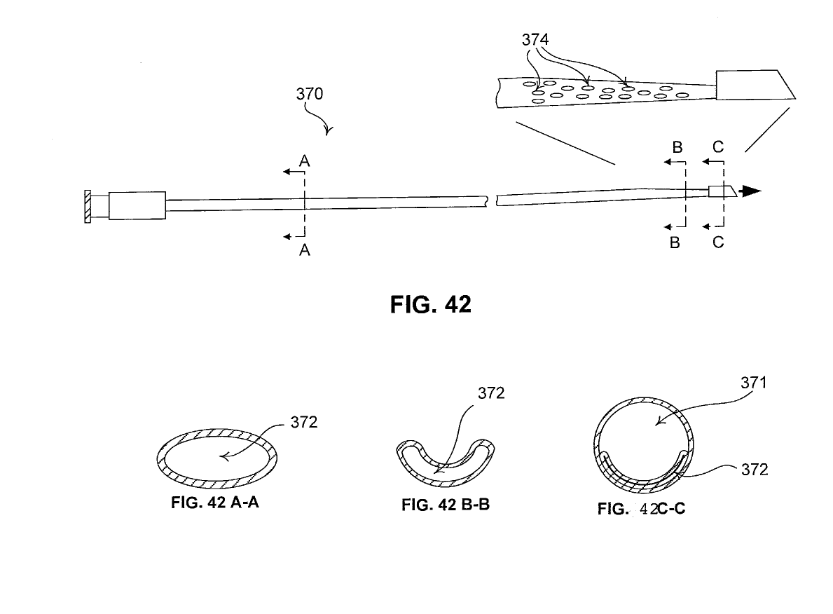

FIG. 42 shows an alternative embodiment of infusion catheter 370. Here, the distal end of the infusion lumen 372 may be closed off, which allows for a smaller distal profile in the region of the rail lumen 371. To allow infused fluid to emerge from the distal region of the catheter, one or more side holes 374 may be provided.

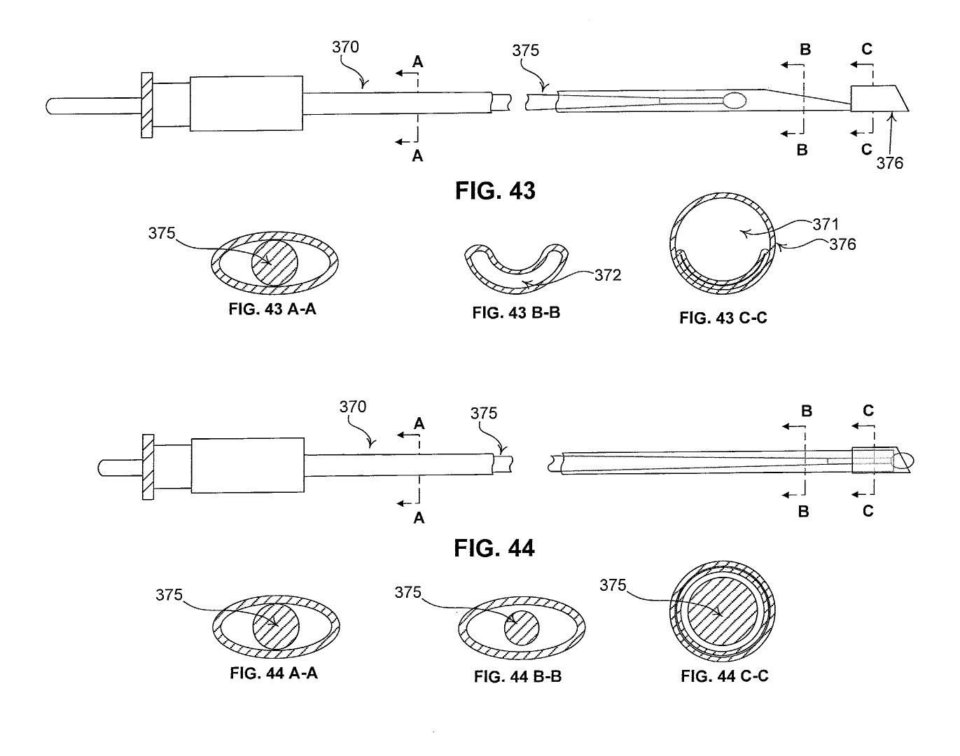

FIGS. 43 and 44 illustrate another alternative embodiment of the infusion catheter 370. The distal end of the infusion lumen 372 is initially closed off, similar to the embodiment of FIG. 42. But here, the infusion lumen 372 can be re-opened upon advancement of a dilator device, such as a stylet 375. Stylet 375 further serves to add stiffness to the infusion catheter 370, which may be useful for advancing the infusion catheter 370 along the guide wire 100, as the guide wire 100 does not provide support for most of the catheter length.

Once the infusion of cooled fluid 297 is desired, the stylet 375 is advanced distally to force open the infusion lumen 372. This is seen in FIG. 44. It may subsequently be removed to clear the infusion lumen 372 for fluid delivery. The open infusion lumen 372 may also be used to deliver a guide wire 100, should that be desirable or necessary in the procedure. Infusion catheter 370 may be made of a polymeric material. Such a material is suitable to being reshaped by the advancement of a stylet 375. The rail lumen 371 of this and above embodiments may be formed with a relatively short metallic tube 376 affixed about the distal portion of the catheter. A metallic tube 376 may also be radiopaque to aid in visualization on x-ray imaging.

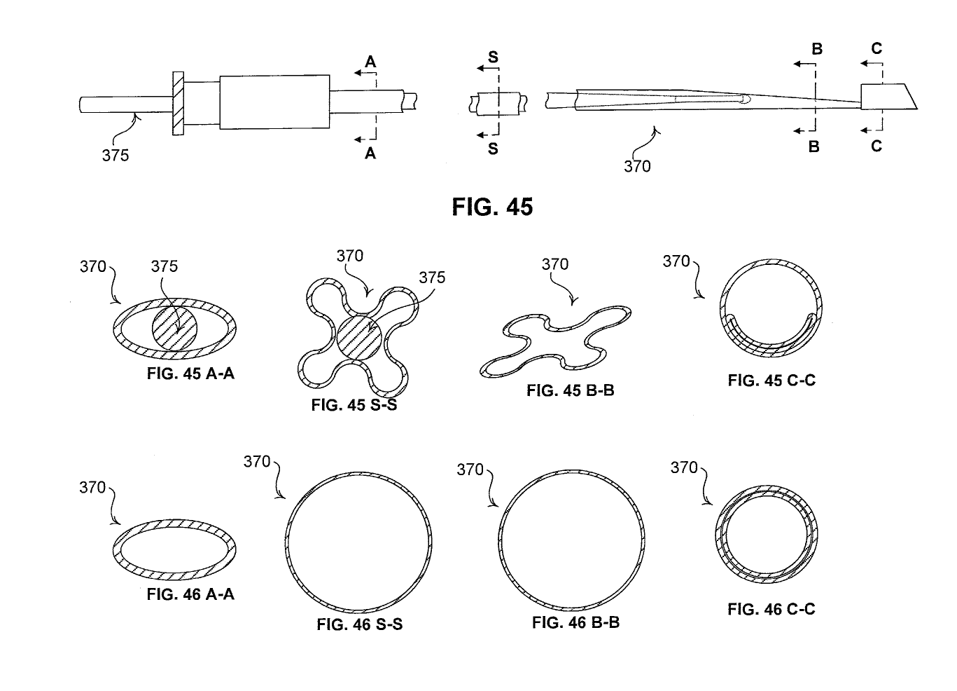

FIG. 45 illustrates yet another embodiment of an infusion catheter 370. Here, some or the entire shaft of the infusion catheter 370 may be formed of an expandable wall. The initial configuration may be non-circular and relatively low profile, as seen in the FIGS. 45S-S and 45B-B. Once infusion of cooled fluid 297 is initiated, the pressure from the infusion may tend to expand out the dimension of the infusion lumen 372, as seen in FIGS. 46S-S and 46B-B. In this manner, when the infusion catheter 370 is positioned next to another device (e.g. a thrombectomy catheter), it may conform to the available space within the constraints of the guide catheter 80.

The infusion catheter 370 embodiments described in connection with FIGS. 42 through 46 may all be utilized in a manner similar to the embodiments described in FIGS. 38 through 41.

Alternative embodiments of thrombus removal catheters and infusion catheter arrangements similar to those described in FIGS. 37, 42, 43, and 45 are now described. Similarly with the above-mentioned embodiments, these embodiments are designed to be space efficient in a relatively small guide catheter, as is typically used in current interventional cardiology procedures. Guide catheter sizes are commonly 6 or 7 French outer diameter. It can be challenging to fit multiple devices, such as catheters and guide wires within the inner diameter of such guide catheters. As such, incorporating non-circular shapes for either the infusion catheter 370 and/or the aspiration/thrombus removal catheter 300 allow for more efficient use of the available cross-sectional area and diameter of relatively small guide catheters 80.

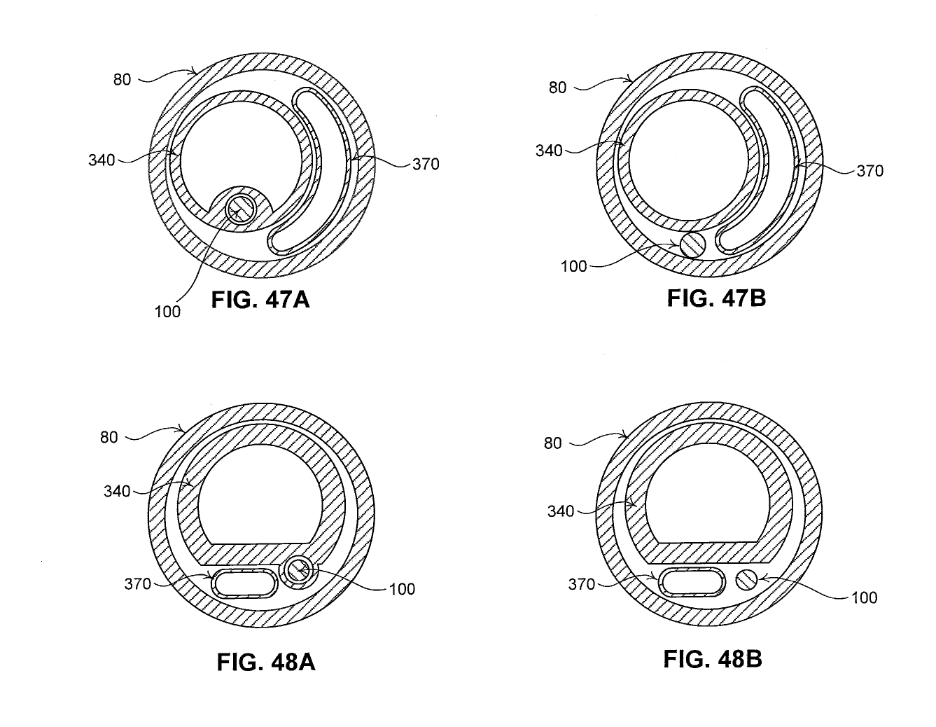

FIGS. 47A and 47B represent cross sectional views of distal and proximal portions, respectively, of one embodiment of infusion catheter 370 and thrombus removal catheter 300. The thrombus removal catheter 300 may incorporate an aspiration lumen 302 and the distal portion may include a shorter guide wire lumen 301 for placement over a guide wire 100, in a monorail or rapid exchange fashion. Infusion catheter 370 may also have a guide wire lumen, but such a lumen may be further distal than illustrated in this figure. Infusion catheter may have a semi-circular infusion lumen, 372 as shown, which may nest about the circular profile of the thrombus removal catheter 300.

FIGS. 48A and 48B show cross sectional views of distal and proximal portions, respectively, of yet another embodiment of infusion catheter 370 and thrombus removal catheter 300. In this embodiment, both the infusion catheter 370 and thrombus removal catheter 300 are non-circular. Thrombus removal catheter 300 has a generally D-shaped cross section in the distal and proximal regions, and may further incorporate a guide wire lumen 301 in the distal portion for tracking over a guide wire 100. In the proximal region, the guide wire 100 may occupy space adjacent the infusion catheter 370 and the thrombus removal catheter 300, within the lumen of guide catheter 80.

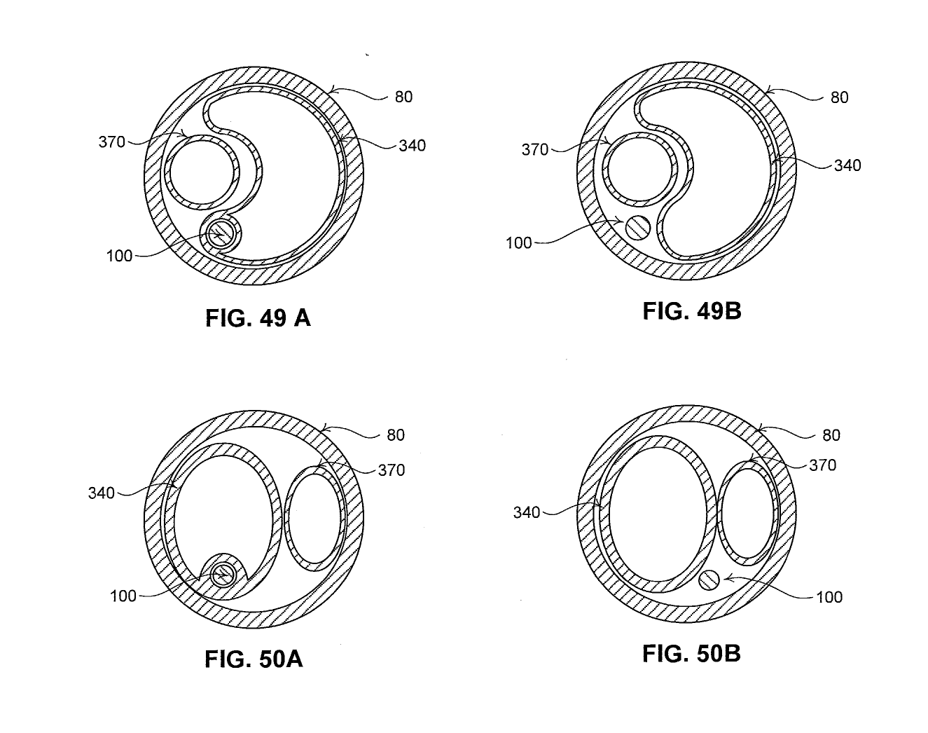

FIGS. 49A and 49B illustrate cross sectional views of distal and proximal portions, respectively, of another embodiment of infusion catheter 370 and thrombus removal catheter 300. In this embodiment, infusion catheter 370 may be relatively circular throughout. A lumen for tracking over a guide wire 100 may also be incorporated for some or all of its length, however, in a preferred embodiment, a short tracking lumen near its distal tip is not seen in this particular cross section. The thrombus removal catheter 300 may have a semi-circular cross sectional shape, which nests about the infusion catheter 370, thereby taking advantage of more of the available cross sectional area within the guide catheter 80.

FIGS. 50A and 50B illustrate yet a further embodiment of infusion catheter 370 and thrombus removal catheter 300, showing distal and proximal portions, respectively. In this embodiment, both infusion catheter 370 and thrombus removal catheter 300 have relatively elliptical cross sectional shapes. The distal portion of the thrombus removal catheter 300 may further incorporate a lumen 301 for tracking over a guide wire 100. While this embodiment does not capture as much of the cross sectional space of the guide catheter 80 as some of the above embodiments, the relatively smooth surfaces of the lumens of each catheter, particularly the thrombus removal catheter 300, may pass fluids more efficiently than less smooth lumens, such as the D-shape of FIGS. 48A and 48B. For example, thrombus particulate may pass more readily through an oval or elliptical cross sectional lumen than a D-shaped lumen.

With reference to FIGS. 51A-51B, temperature recordings in bench tests are shown. The bench testing was performed to demonstrate that localized cooling could be achieved by infusing cooled fluid solution through catheters. The bench model involved a simulated femoral artery, aorta, and coronary arteries. Fluid heated to 37° C. was pumped through the vessels to simulate blood flow and the setup was immersed into a 37° C. bath to simulate the body. Cold fluid was provided by a refrigerated bag of saline routed by a line set through a pump and through an ice bath. The line set was connected to the hub of an infusion catheter. The infusion catheter was tracked through the femoral artery, up the aorta, and into the simulated coronary vessel. Temperature probes were placed within the simulated coronary artery, as well as in the saline bag where the cold fluid originated, in the heated bath that contained the model, and in the hub of the infusion catheter. As cold fluid was infused through the infusion catheter, temperature recordings were made in the coronary artery (labeled coronary), fluid reservoir (labeled saline bag), at the hub of the infusion catheter (labeled hub), and in the ice bath (labeled fixture bath or line bath). In FIG. 51A, the cold fluid infusion rate was 20 mL/min in a simulated open vessel with competing blood flow. In FIG. 51B, the cold fluid infusion rate was 12 mL/min in a simulated occluded vessel with no competing blood flow. In FIG. 51C, the initial cold fluid infusion rate was 10 mL/min, followed by a pause of infusion, followed by a resumption of infusion at a rate of 20 mL/min.

FIG. 51A shows temperature measurements with a cold fluid infusion rate of 20 mL/min in an open (non-occluded) vessel, and FIG. 51B shows temperature measurements with a cold fluid infusion rate of 12 mL/min in a closed (occluded) vessel. In the coronary artery (i.e., target region), the average temperature reduction from normal body temperature (37° C.) was found to be about 3.3° C. in the open vessel model and about 4.7° C. in the closed vessel model when the cold fluid temperature at the hub of the infusion catheter was measured to be approximately 12° C. Cooling was found to be very rapid once infusion was started and the desired temperature drop generally occurred in under a minute. Re-warming in the blood vessel after the infusion was discontinued was also rapid. In general, once the temperature plateaued in the simulated coronary artery, continued infusion appeared to just maintain and stabilize the temperature and thus the temperature did not continue to decrease further over time. There also appeared to be a consistent correlation between the temperature measured in the hub of the infusion catheter and the temperature measured in the simulated coronary for a given flow rate.

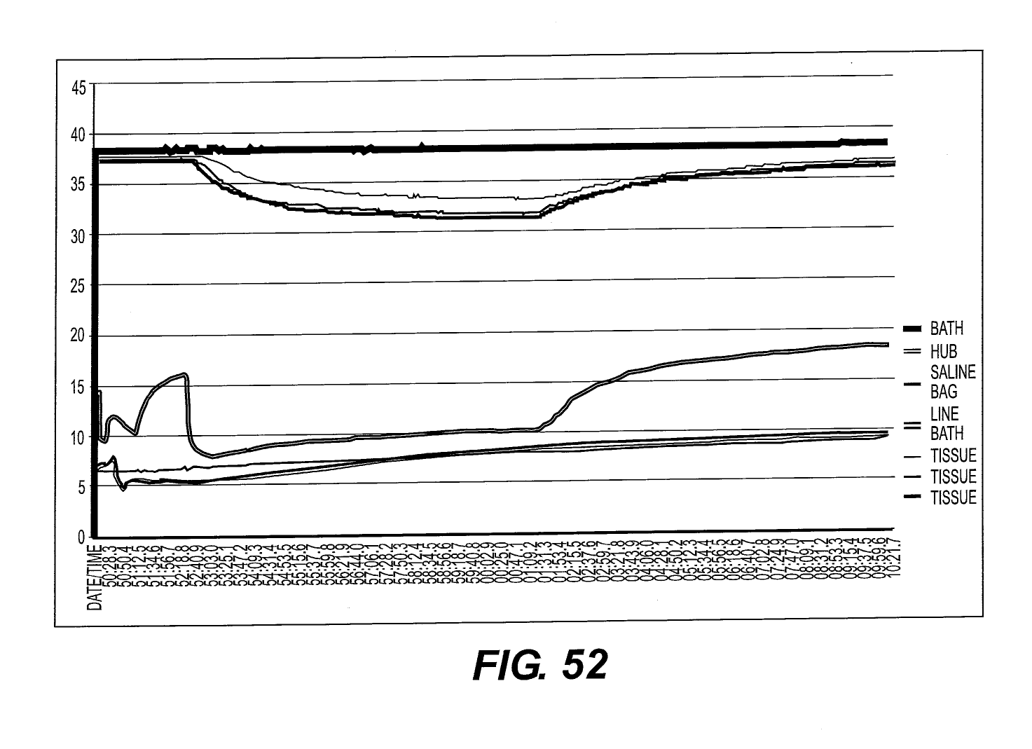

With reference to FIG. 52, temperature recordings in a cadaveric animal tissue model are shown. This testing was performed to demonstrate that localized cooling could be achieved in myocardium tissue by infusing cooled solution through catheters. FIG. 52 shows temperature measurements plotted over time in a tissue model. The model consisted of a cadaveric porcine heart. Fluid heated to 37° C. was pumped through the vessels of the heart to simulate blood flow and the setup was immersed into a 37° C. bath to simulate the body. The infusion catheter was tracked into the porcine coronary vessel and cold fluid was infused through the infusion catheter. Three temperature probes were placed within the myocardial tissue at a position downstream from the infusion, as well as in the saline bag where the cold fluid originated, the heated bath where the tissue was immersed, and in the hub of the infusion catheter. A probe labeled ‘Line Bath’ measured the temperature in an ice bath where a portion of the infusion tubing between the pump and the catheter was immersed to further cool solution running through it. FIG. 52 shows cold fluid infusion rate of 20 mL/min in an open (non-occluded) vessel and the corresponding temperature measurements. Although cooling and warming were slower in the tissue model than in the bench model, much of the conclusions made in the bench model also applied to the tissue model.

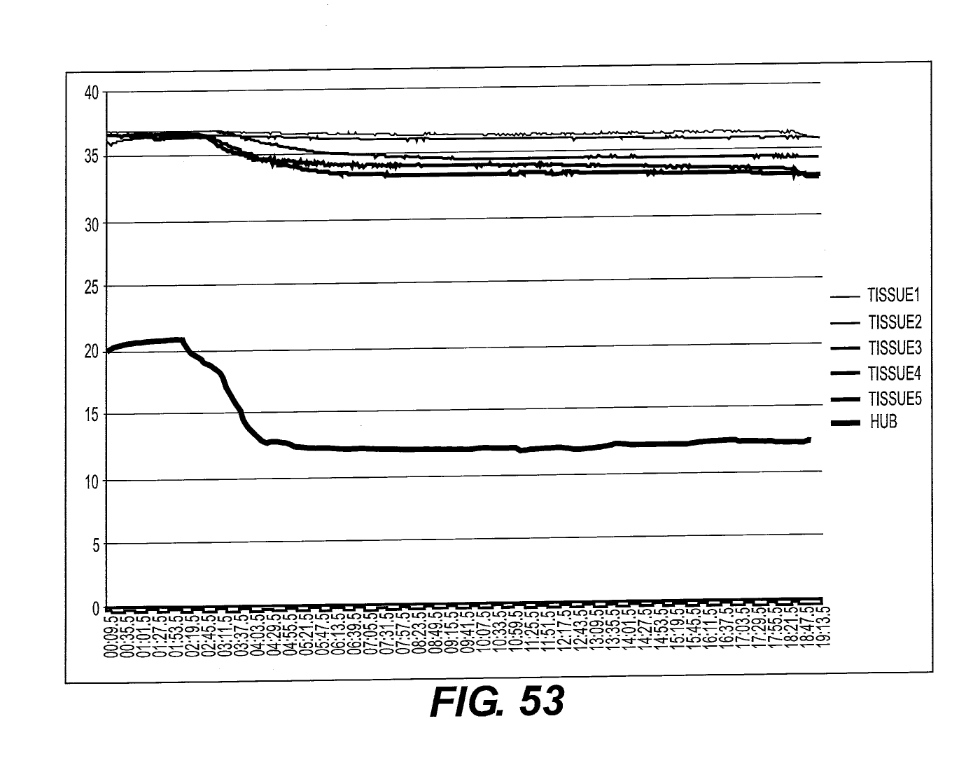

With reference to FIG. 53, temperature recordings in a live animal tissue model are shown. This testing in a porcine model was also performed to demonstrate that localized cooling of myocardial tissue could be achieved by infusing cooled solution through catheters in vivo. FIG. 53 shows temperature measurements plotted over time in a live porcine model. The probes labeled ‘Tissue’ on the graph represent the temperature measured over time of five temperature probes that were placed approximately 10 millimeters into the myocardium of a 77 kg pig. A sternotomy was used to access the heart and place the temperature probes. Probe locations ranged from proximal to the infusion catheter tip to distal to the infusion catheter tip. The probe titled ‘Hub’ represents the temperature measured from the hub of the infusion (balloon) catheter during infusion. The infusion catheter consisted of a Cordis Savvy PTA 0.018″ peripheral balloon catheter (3.5×20 mm) and was placed into the mid to distal LAD and inflated to 2 ATM prior to infusion. The temperature data logger was started to record baseline data. Occlusion was confirmed and infusion started gradually through the balloon lumen (approx. 1 min to reach the full infusion rate of 15 mL/min). Again, although cooling and warming were slower in the tissue model than the bench model, much of the conclusions made in the bench and tissue models also applied to the animal model. Also, it was found that tissue upstream of the tip of the infusion catheter did not cool as efficiently and sometimes showed little to no cooling.

From the foregoing, it will be apparent to those skilled in the art that the present invention provides various embodiments of devices and methods that mitigate reperfusion injury (RI) in a clinically practical manner so as to avoid significantly increasing time to reperfusion. In general, these systems and methods involve an antegrade approach to deliver a fluid to the myocardium at risk of RI before, during and after reperfusion is established by a percutaneous coronary intervention such as stenting. A variety of devices, fluids, and procedural steps are disclosed to mitigate RI by, for example: pre-conditioning (e.g., reducing the temperature of) the affected myocardium; controlling reperfusion dynamics (e.g., flow rate, oxygenation, flushing, buffering, etc.) to the affected myocardium, and/or post-conditioning the affected myocardium. These embodiments may be used to treat other clinical conditions, as well as other anatomical sites.

We claim:

1. A method of treating a patient, comprising:
a) advancing a catheter across an occlusion in a coronary artery;
b) infusing a chilled liquid via the catheter distal of the occlusion before reperfusion;
c) aspirating the occlusion to establish reperfusion while continuing to infuse the chilled fluid during reperfusion;
d) retracting the catheter proximally and/while continuing to infuse the chilled fluid proximal of the occlusion; and
e) dilating the occlusion while continuing to infuse the chilled fluid.

2. A method as in claim 1, wherein the chilled liquid is infused at a first flow rate before the occlusion is aspirated and a second flow rate after the occlusion is aspirated, wherein the first flow rate is less than the second flow rate.

3. A method as in claim 1, wherein the chilled liquid is infused at a first flow rate before the occlusion is dilated and a second flow rate after the occlusion is dilated, wherein the first flow rate is less than the second flow rate.

4. A method as in claim 1 wherein the chilled liquid is infused before aspiration and continuously during aspiration.

5. A method as in claim 4 wherein the chilled liquid is infused before dilation and continuously during dilation.

6. A method as in claim 5 wherein the chilled liquid is infused after dilation.

7. A method as in claim 1, wherein the chilled liquid is provided via an insulated line set connecting a source of pressurized chilled liquid to a proximal end of the catheter, and wherein the pressurized chilled liquid is recirculated via the insulated line set through the source of pressurized chilled liquid to maintain a constant temperature of chilled fluid to the catheter.

8. A method as in claim 1, wherein the chilled liquid is provided via an insulated line set connecting a source of pressurized chilled liquid to a proximal end of the catheter, and wherein the pressurized chilled liquid is purged from the line set proximate the proximal end of the catheter before infusion.

9. A method as in claim 1, wherein the chilled liquid is provided via an insulated line set connecting a source of pressurized chilled liquid to a proximal end of the catheter, wherein a temperature sensor is provided in contact with the chilled liquid proximate the proximal end of the catheter, wherein the temperature sensor is electrically connected to the source of pressurized chilled liquid, and wherein the temperature of the pressurized chilled liquid is controlled by closed-loop feedback from the temperature sensor as a function of infusion flow rate of the chilled liquid.

10. A method of treating a patient, comprising:
a) placing a guide wire across an occlusion in a coronary artery;
b) advancing a first catheter over the guide wire and across the occlusion;
c) infusing a chilled liquid via the first catheter distal of the occlusion before reperfusion;
d) disengaging the first catheter from the guide wire while leaving the guide wire across the occlusion; and
e) advancing a second catheter over the guide wire while continuing to infuse chilled liquid.

11. A method as in claim 10, wherein the second catheter is a thrombus removal catheter.

12. A method as in claim 10, wherein the second catheter is a balloon dilation catheter.

13. A method as in claim 10, wherein the second catheter is a stent delivery catheter.

14. A method as in claim 10, wherein the first catheter has a short guide wire lumen with a proximal end positioned distal of the occlusion, and wherein the first catheter is disengaged from the guide wire by moving the first catheter distal relative to the guide wire while the guide wire remains across the occlusion.

15. A method as in claim 14, wherein the guide wire, first catheter and second catheter are disposed in a round lumen of a guide catheter, and wherein the first and second catheters have non-round cross-sectional profiles in the lumen of the guide catheter.

16. A method as in claim 10, wherein the chilled liquid is infused at an initial flow rate and a subsequent flow rate, and wherein the initial flow rate is less than the subsequent flow rate.

17. A method as in claim 10, wherein the chilled liquid is provided via an insulated line set connecting a source of pressurized chilled liquid to a proximal end of the first catheter, and wherein the pressurized chilled liquid is recirculated via the insulated line set through the source of pressurized chilled liquid to maintain a constant temperature of chilled fluid to the first catheter.

18. A method as in claim 10, wherein the chilled liquid is provided via an insulated line set connecting a source of pressurized chilled liquid to a proximal end of the first catheter, and wherein the pressurized chilled liquid is purged from the line set proximate the proximal end of the first catheter before infusion.

19. A method as in claim 10, wherein the chilled liquid is provided via an insulated line set connecting a source of pressurized chilled liquid to a proximal end of the first catheter, wherein a temperature sensor is provided in contact with the chilled liquid proximate the proximal end of the first catheter, wherein the temperature sensor is electrically connected to the source of pressurized chilled liquid, and wherein the temperature of the pressurized chilled liquid is controlled by closed-loop feedback from the temperature sensor as a function of infusion flow rate of the chilled liquid.

20. A system for treating a patient having an occlusion in a coronary artery, comprising:
a) an external source of pressurized chilled liquid;
b) a guide catheter configured to extend intravascularly to the coronary artery;
c) a guide wire configured to extend through the guide catheter, through the coronary artery, and across the occlusion; and
d) an infusion catheter configured to be advanced through the guide catheter, over the guide wire and across the occlusion, the infusion catheter having a proximal end connect via a fluid line to the external source of pressurized chilled liquid, wherein the infusion catheter includes an infusion lumen configured to deliver the chilled liquid distal of the occlusion, and a guide wire lumen configured to disengage from the guide wire while the guide wire and infusion catheter remain across the occlusion.

21. A system as in claim 20 wherein the guide wire lumen of the infusion catheter has a proximal end configured to be disposed distal of the occlusion such that the guide wire lumen can disengage from the guide wire by advancing the infusion catheter relative to the guide wire.

22. A system as in claim 21 wherein the guide wire lumen has a length of less than 5 cm.

23. A system as in claim 21 wherein the guide wire lumen has a length of less than 2.5 cm.

24. A system as in claim 21 wherein the guide wire lumen has a length of about 1 cm.

25. A system as in claim 21, further comprising a second catheter configured to be advanced through the guide catheter alongside the infusion catheter and over the guide wire.

26. A system as in claim 25 wherein the guide catheter has a round lumen extending therethrough and the second catheter has an other-than-round profile in the guide catheter to accommodate the infusion catheter alongside.
